SEQUENCE LISTING (20 sequences)

SEQ ID NO: 1 - RNA, 25 nt, Artificial Sequence
  siEphA2
gcaaggaagugguacugcuggacuu

SEQ ID NO: 2 - RNA, 19 nt, Artificial Sequence
  siTrkA 1
gaaccugacugagcucuac

SEQ ID NO: 3 - RNA, 19 nt, Artificial Sequence
  siTrkA 2
uggagucucucuccuggaa

SEQ ID NO: 4 - RNA, 19 nt, Artificial Sequence
  siTrkA
gcugcagugucaugggcaa

SEQ ID NO: 5 - protein, 8 aa, Artificial Sequence
  Fragment of protein
IGDFGMSR

SEQ ID NO: 6 - protein, 10 aa, Artificial Sequence
  fragment of protein
WELGEGAFGK

SEQ ID NO: 7 - protein, 11 aa, Artificial Sequence
  fragment of protein
NCLVGQGLVVK

SEQ ID NO: 8 - protein, 9 aa, Artificial Sequence
  fragment of protein
DIYSTDYYR

SEQ ID NO: 9 - protein, 11 aa, Artificial Sequence
  fragment of protein
ACPPEVYAIMR

SEQ ID NO: 10 - protein, 10 aa, Artificial Sequence
  fragment of protein
WMPPESILYR

SEQ ID NO: 11 - protein, 12 aa, Artificial Sequence
  fragment of protein
FVAPDAFHFTPR

SEQ ID NO: 12 - protein, 13 aa, Artificial Sequence
  fragment of protein
WEEEGLGGVPEQK

SEQ ID NO: 13 - protein, 15 aa, Artificial Sequence
  fragment of protein
VFLAECHNLLPEQDK

SEQ ID NO: 14 - protein, 15 aa, Artificial Sequence
  fragment of protein
EAELLTMLQHQHIVR

SEQ ID NO: 15 - protein, 20 aa, Artificial Sequence
  fragment of protein
GLEQAGWILTELEQSATVMK

SEQ ID NO: 16 - protein, 24 aa, Artificial Sequence
  fragment of protein
TVQGLSLQELVLSGNPLHCSCALR

SEQ ID NO: 17 - protein, 9 aa, Artificial Sequence
  fragment of protein
IYSFGLGGR

SEQ ID NO: 18 - protein, 10 aa, Artificial Sequence
  fragment of protein
FADIVSILDK

SEQ ID NO: 19 - protein, 10 aa, Artificial Sequence
  fragment of protein
TVSEWLESIK

SEQ ID NO: 20 - protein, 13 aa, Artificial Sequence
  fragment of protein
AANILVGENLVCK

(19) United States
(12) Patent Application Publication
(10) Pub. No.: US 2017/0258871 A1
(43) Pub. Date: Sep. 14, 2017

(54) COMBINED USE OF A TRKA INHIBITOR AND AN EPHA2 INHIBITOR FOR USING IN THE TREATMENT OF SOLID CANCERS, AND METHOD FOR THE PROGNOSIS OF SURVIVAL OF A PATIENT WHO HAS A SOLID CANCER

(71) Applicants: UNIVERSITE DES SCIENCES ET TECHNOLOGIES DE LILLE, Villeneuve D'ascq (FR); INSERM (INSTITUT NATIONAL DE LA SANTE ET RESEARCHE MEDECALE), Paris (FR)

(72) Inventors: Robert Toillon, Bauvin (FR); Xuefen Le Bourhis, Chereng (FR); Cyril Corbet, Hellemmes (FR); Leo Aubert, Lille (FR); Matthieu Guilbert, Pont A Marcq (FR)

(73) Assignees: UNIVERSITE DES SCIENCES ET TECHNOLOGIES DE LILLE, Villeneuve D'ascq (FR); INSERM (INSTITUT NATIONAL DE LA SANTE ET DE LA RECHERCHE MEDICALE), Paris (FR)

(21) Appl. No.: 15/511,739

(22) PCT Filed: Sep. 7, 2015
(86) PCT No.: PCT/FR2015/052368

§ 371 Date: Mar. 16, 2017

(30) Foreign Application Priority Data
Sep. 16, 2014 (FR) 14 58741

(51) Int. Cl. A61K 38/17; G01N 33/574; A61K 31/553

(52) U.S. Cl. CPC A61K 38/177; A61K 31/553; G01N 33/57492; G01N 33/57415; G01N 33/57434; G01N 33/57407; G01N 2333/705

(57) ABSTRACT

The invention relates to an EphA2 inhibitor for the use thereof in the treatment of solid cancer treated with a TrkA inhibitor, and a TrkA inhibitor for the use thereof in the treatment of solid cancer treated with an EpbA2 inhibitor. The invention also relates to a method for the prognosis of survival of a patient who has a solid cancer, comprising a step of detecting the expression of TrkA and EphA2 in a biological sample of the patient, the co-expression of TrkA and EphA2 being associated with a poor prognosis of survival of the patient.

The invention relates to the combined use of a TrkA inhibitor and an EphA2 inhibitor for using in the treatment of solid cancers. The invention also relates to a method for the prognosis of survival of a patient who has a solid cancer.

The progress of a cancer involves many steps, dependent in particular on the size of the primary tumour but also on the capacity of the tumour cells to metastasize in organs remote from the primary tumour. Many reasons can explain the significant number of failures in therapeutic treatment of cancers.

In many cases, therapeutic failure is associated with activation of signal transduction on the tyrosine kinase activity receptors (1).

The receptors with tyrosine kinase activity (RTK), also known as growth factor receptors, constitute a subfamily of the kinase proteins, acting specifically on protein tyrosine residues. These transmembrane glycoproteins comprise a highly variable extracellular region capable of binding the ligand, a transmembrane region enabling anchoring in the cellular membrane and an intracellular (cytoplasmic) region which includes the tyrosine kinase activity and enables signal transduction within the cell. These RTK can be categorized in several subclasses according to the properties of the extracellular region of the tyrosine kinase receptors.

Hence, the emergence of anti-tumour therapies targeting growth factors, the receptors thereof (tyrosine kinase activity receptors) or the effectors of the signalling thereof, has enabled the development of anti-cancer drugs.

In this context, many studies have investigated the role of Nerve Growth Factor (NGF) in different types of cancer, in particular in breast cancer. NGF is the prototypical member of the family of neurotrophins which act via the TrkA receptors (tropomyosin receptor kinase A) and p75^{NTR} receptors (p75 neurotrophin receptor). NGF has been shown to play a role in non-neuronal cells, in particular in several types of cancer cells. For example, it has been shown that NGF could promote tumour development in preclinical mouse models (2).

More recent studies have been able to show that the precursor of NGF, proNGF, is also expressed, in a specific manner, in different types of cancer, in particular breast cancer (see for example application US 2009068200 A1), and that the TrkA receptor is not only activated by NGF but also by its precursor, proNGF. It has also been demonstrated that the expression of proNGF in mammalian tumours is correlated with lymph node invasion. In addition, proNGF could increase the migratory and invasive capabilities of breast cancer cells in vitro (3).

It has recently been shown by researchers that the pro-invasive action of proNGF in mammalian tumour cells is linked to its binding on sortilin, a transmembrane receptor with a Vps10p domain, and to TrkA receptor activation. Phosphorylation of TrkA can induce the proteins Src and Akt, which play a role in cancer development (3).

Research undertaken to date therefore suggests that the proNGF/TrkA axis acts in a significant manner in the tumour progression of solid cancers (carcinomas or sarcomas), in particular for breast cancer.

However, it has been shown that treatments using TrkA inhibitors, such as lestaurtinib and the derivatives thereof, only have limited efficacy, as demonstrated by the clinical studies performed (4). Treatments using TrkA inhibitors are therefore not used for solid tumours.

Thus there is still a need for new treatments of solid cancers, in particular breast cancer.

However, after much research, the inventors have discovered that the use of a TrkA inhibitor and of an EphA2 inhibitor together allows effective treatment of certain forms of cancers, in particular solid cancers and especially breast cancer. Indeed, the inventors have successfully demonstrated, in a surprising manner, a signalling platform in breast cancer cells that is at the origin of a resistance to commonly used TrkA tyrosine kinase inhibitors.

It has also been demonstrated that, in cancer cells from solid cancers, particularly in breast cancer cells, the TrkA receptor associates directly with the EphA2 receptor under the effect of binding of proNGF on sortilin, and that the TrkA/EphA2 complex is responsible for activation of a signalling, independent of the phosphorylation of TrkA which is to date known and targeted by lestaurtinib. The simultaneous detection of TrkA and Eph2 also confirms the importance of signalling induced by said complex. Indeed, the presence of the complex is associated with a significant reduction in the overall survival of the patients.

The invention therefore relates to an EphA2 inhibitor for use in the treatment of solid cancer treated by a TrkA inhibitor.

The invention also relates to a TrkA inhibitor for use in the treatment of solid cancer treated by an EphA2 inhibitor.

The use of the two inhibitors makes it possible to inhibit, in an effective and synergistic manner, distinct signalling pathways induced under the effect of proNGF.

The TrkA inhibitor inhibits signalling pathways, dependent on phosphorylation of TrkA, which are known to date. As previously mentioned, it has indeed already been demonstrated that proNGF, by binding on sortilin, causes the activation of the TrkA receptor, in turn inducing signalling pathways that are dependent on the phosphorylation thereof. Here, it has been confirmed by the inventors that phosphorylation of TrkA allows, in particular, activation of the protein Akt, participating in a significant manner in cancer cell invasion.

As previously mentioned, TrkA is one of the Trk kinases, a subfamily of the RTK. The Trk kinases comprise three isoforms having a strong homology: TrkA, TrkB and TrkC, and are activated by growth factors, the neurotrophins. TrkA is usually activated by NGF, but also by its precursor, proNGF.

According to the invention, “TrkA inhibitor”, shall mean a TrkA antagonist, an inhibitor of TrkA receptor expression, an inhibitor of Trka tyrosine kinase activity or a molecule preventing the binding of signalling adapter molecules. Preferably, a TrkA inhibitor refers to an inhibitor of Trka tyrosine kinase activity or a TrkA antagonist.

According to a first embodiment, in which in particular the TrkA inhibitor is a Trka tyrosine kinase activity inhibitor, said inhibitor is a small organic molecule with low molecular weight (natural or otherwise).

The term “small organic molecule” refers to a molecule (natural or otherwise) having a size comparable to the organic molecules generally used in the pharmaceutical domain. This term excludes biological macromolecules (for example proteins, nucleic acids, etc.). Preferably, a small organic molecule according to the invention has a size less than approximately 10,000 Da, preferably less than 5000 Da, more preferably less than 2000 Da and yet more preferably less than approximately 1000 Da.

Such TrkA inhibitors are well known to a person skilled in the art (see for example, Wang et al. (5), or WO 2008/052062 the content of which is incorporated by reference), or can easily be generated by a person skilled in the art.

Preferably, the TrkA inhibitor according to the invention is selected from K252a (see for example Lelkes et al. (6)) and CEP-701 (see for example Ruggeri B et al. (7)).

According to a particular embodiment, the TrkA inhibitor is an alkyne derivative (see for example Wang et al. (5), WO2006044823, Cee V J et al. (8)).

According to a particular embodiment, the TrkA inhibitor is a pyrazole derivative (see for example Wang et al. (5)), and particularly the pyrazolopyrimidine derivatives (see for example WO2005103010, WO2006082392, WO2006117560), or a purine derivative (see for example WO2006087530, WO2006087538, Hudkins R L et al. (9)).

According to a particular embodiment, the TrkA inhibitor is a pyrazole urea derivative (see for example Wang et al. (5), WO2007064872, WO2007059202, WO2005110994).

According to a particular embodiment the TrkA inhibitor is an aminothiazole derivative (see for example Wang et al. (5), Das J et al. (10); WO200250071; Kim S H et al. (11))

According to a particular embodiment, the TrkA inhibitor is a pyrrolotriazine derivative (see for example Wang et al. (5), WO2007061882; Ghose A K et al. (12), WO2008057994).

According to a particular embodiment, the TrkA inhibitor is an indolocarbazole derivative (see for example Wang et al. (5)) such as CEP-701 or lestaurtinib (see for example Ruggeri B et al. (7)) or the analogues (3′S)-epi-K-252a (see for example, Gingrich D E et al. (13)) or the 7-oxo-indenopyrrolocarbazoles substituted in position 13 (see for example Tripathy R et al. (14)).

According to a particular embodiment, the TrkA inhibitor is a condensed cyclic pyrazolyl derivative (see for example Wang et al. (5), JP2003231687).

According to a particular embodiment, the TrkA inhibitor is a tetrahydropyrrolopyrazole derivative (see for example Wang et al. (5)), comprising in particular a bicyclic 1,4,5,6-tetrahydropyrrolo[3,4-c]pyrazole such as PHA-739358 (see for example, WO200505427; Fancelli D et al. (17)).

According to a particular embodiment, the TrkA inhibitor is an imidazopyridazine derivative (see for example Wang et al. (5), WO200852734).

According to a particular embodiment, the TrkA inhibitor is an isothiazole derivative (see for example Wang et al. (5), WO2004011461; Lippa B et al. (18)).

According to a particular embodiment, the TrkA inhibitor is a pyrrolopyrimidine derivative (see for example Wang et al. (5)), such as CE-245677 (see for example, WO2004056830, WO2005116035).

According to a particular embodiment, the TrkA inhibitor is an azaindole derivative (see for example Wang et al. (5), WO2008080001, WO2008063888).

According to a particular embodiment, the TrkA inhibitor is GNF-5837 (see for example Albaugh et al. (19)).

According to a particular embodiment, the TrkA inhibitor is (2E)-3-[3,5-bis(11,1-dimethylethyl)-4-hydroxyphenyl]-2-cyano-2-propenethioamide, known under the name AG 789.

According to another embodiment, said inhibitor according to the invention is a TrkA antagonist. An antagonist is typically any substance, simple or complex compound, of natural or synthetic origin, which opposes the activation of the TrkA receptor and which opposes in particular the induction of the biological response that is obtained with a natural ligand of said receptor. In general, such an antagonist alters the signalling or the recruitment of TrkA partners in the cell.

According to one example, the TrkA antagonist is an anti-TrkA antibody which neutralizes TrkA or an anti-TrkA fragment of same which neutralizes TrkA.

Anti-TrkA antibodies are known to a person skilled in the art. Reference can be made for example to application WO 2013183032 or application WO 2009098238A.

Antibodies against TrkA can be produced according to methods known to a person skilled in the art, in particular by administering an antigen or an epitope to a selected host animal, for example a pig, cow, horse, rabbit, goat, sheep or even a mouse. Different adjuvants known in the prior art can be used to improve the production of antibodies. Although the antibody according to the invention can be a polyclonal antibody, a monoclonal antibody is preferred. Monoclonal antibodies against TrkA can be prepared and isolated by any appropriate method, for example flowing the technique of hybridoma initially described by Kohler and Milstein (1975) (20); the technique of EBV hybridoma (Cole et al. 1985) (21), etc. Alternatively, methods described for the production of single-chain antibodies (see for example patent U.S. Pat. No. 4,946,778) can be adapted to produce antibodies with an anti-TrkA single-chain.

Antagonists that can be used according to the invention also include antibody fragments, including but not limited to, F(ab')₂ fragments able to be generated by digestion using the pepsin of an immunoglobulin, Fab fragments able to be generated by production of the disulfide bridges of F(ab')₂ fragments, and scFv fragments. Alternatively, Fab and/or scFv expression libraries can be produced to allow rapid identification of the fragments having the desired specificity for TrkA.

Humanized anti-TrkA antibodies and antibody fragments can also be prepared according to techniques known to a person skilled in the art. A “humanized antibody” refers to a human immunoglobulin (receiver antibody) in which the residues of a hypervariable region (CDRs) are replaced by residues of a hypervariable region of a non-human species (donor antibody) such as a mouse, rat, rabbit etc., which has the desired specificity, affinity and capability. According to some examples, FR residues of human immunoglobulin are replaced by corresponding non-human residues. In addition, humanized antibodies can include residues not found in the receiver or donor antibodies. These modifications are made to improve the performance of the antibodies. Humanized antibodies will possibly include at least one portion from a constant region of an immunoglobulin (Fe), typically that of a human immunoglobulin. Methods for producing humanized antibodies are described, for example, by Winter (patent U.S. Pat. No. 5,225,539) and Boss (Celltech, patent U.S. Pat. No. 4,816,397).

For this invention, the neutralizing antibodies of TrkA are selected.

In another embodiment, the TrkA antagonist is selected from the aptamers. The aptamers are a class of molecules which represent an alternative to antibodies in terms of molecular recognition. The aptamers are oligonucleotides or oligopeptide sequences having the ability to recognize almost any class of target molecule with a high affinity and specificity. Such ligands can be isolated, for example, using a SELEX system (Systematic Evolution of Ligands by EXponential enrichment) from a library of randomized sequences, as described by Tuerk C. and Gold L. (22). The library of randomized sequences can be obtained by chemical combinatorial DNA synthesis. Each member in this library is a linear oligomer, possibly chemically modified, with single sequence.

For this invention, neutralizing aptamers of TrkA are selected.

According to another example, the TrkA antagonist is a natural ligand of TrkA having been modified in such a way as to prevent the binding of a natural TrkA ligand. Natural TrkA ligands are, for example, Nerve Growth Factor (NGF), the precursor thereof (proNGF) and neurotrophin 3 (NT3) (23). A person skilled in the art knows how to modify natural ligands of said receptor in such a way as to produce antagonist molecules.

Although the embodiment according to which the TrkA inhibitor is a TrkA antagonist is preferred, another embodiment according to the invention relates to an inhibitor of TrkA receptor expression (i.e. of the TrkA receptor gene).

Small inhibitory RNA (siRNA) can be used according to the invention as TrkA receptor suppression gene inhibitors. The expression of the gene for the TrkA receptor can be reduced by bringing a subject or a cell into contact with small fragments of double-stranded RNA (dsRNA), or a vector or a genetic construction inducing the production of small fragments of double-stranded RNA, in such a way that the expression of the TrkA receptor gene is specifically inhibited (i.e. RNA interference or RNAi). The methods for selecting a dsRNA or a vector coding for dsRNA are known from the prior art for genes for which the sequence is known (see for example applications WO 01/36646, and WO 01/68836).

Ribozymes can also be used according to the invention as inhibitors of the expression of the TrkA receptor gene. Ribozymes are enzymatic RNA molecules that are capable of catalysing RNA cleavage. The mechanism of the action of ribozymes involves the hybridization of a specific sequence of the ribozyme molecule to the complementary RNA target, followed by an endonucleolytic cleavage. The hairpin or hammerhead ribozymes which specifically and effectively catalyse the endonucleolytic cleavage of the mRNA sequences of the TrkA receptor can therefore be used according to the invention. The specific cleaving sites in any target RNA can be initially identified by scanning the target molecule for the cleavage sites, which generally comprise the following sequences: GUA, GUU and GUC. Once identified, short RNA sequences having between 15 and 20 ribonucleotides corresponding to the target region of the gene containing the cleavage site can be evaluated in order to predict the structural characteristics, such as the secondary structure, which may render the oligonucleotide sequence non-appropriate. The appropriate character of the target can also be evaluated by testing its accessibility to hybridization with complimentary oligonucleotides by using, for example, ribonuclease protection techniques.

The antisense siRNA oligonucleotides and the ribozymes able to be used as inhibitors for the expression of the TrkA receptor gene can be prepared according to known methods. Chemical synthesis methods can be used, such as for example solid-phase phosphoramidite synthesis. Alternatively, antisense RNA molecules can be generated by in vitro or in vivo transcription of the DNA sequences coding the RNA molecule. Such DNA sequences can be incorporated in a wide variety of vectors which include appropriate RNA polymerase promoters such as the polymerase promoters T7 or SP6. Modifications can be introduced into the oligonucleotides according to the invention, in order to increase their intracellular stability and half-life. Modifications that can be made include, but are not limited to, the addition of flanking regions of ribonucleotides or deoxyribonucleotide at the 5′ and/or 3′ end of the molecule, or the use of phosphorothioate or of 2′-O-methyl rather than the phosphodiesterase bonds in the oligonucleotide backbone.

The siRNA and ribozymes of the invention can be produced in vivo alone or in association with the vector. “Vector” shall mean any vehicle capable of facilitating the transfer of siRNA or of ribozyme to the cells, and preferably cells expressing the TrkA receptor. The vector preferably transports nucleic acid to the cells with a reduced degradation compared to the degradation which would result in the absence of the vector. In general, the vectors that can be used according to the invention include, but are not limited to, plasmids, phagemids, viruses and other vehicles derived from viral or bacterial sources and having been manipulated by insertion or incorporation of siRNA or of the ribozyme nucleic acid sequences. Such vectors are well known to a person skilled in the art.

Finally, it is possible to use a molecule preventing the binding of signalling adapter molecules as a TrkA inhibitor. “Signalling adapter molecule” shall mean molecules which interact with the TrkA receptor via the interaction domains in order to trigger intracellular signals. Signalling adapter molecules of the TrkA receptor are for example the proteins shc, FRS2, PLCgamma1 and Grb2. These inhibitors can be peptides for example, generally obtained by synthesis of all or part of a signalling molecule, which would have a negative dominant effect on the bonds of cellular adapters. These molecules can also be organic mimetics, i.e. non-peptide analogues, the conformation of which enables recognition of the TrkA receptor sequence in order to bind there and by competition prevent the binding of the signalling partner (56). Through their bonding, these inhibitors can also prevent activation of the TrkA receptor by inhibiting the changes in conformation necessary for TrkA activation (for example, by an allosteric antagonist such as AR-786 developed by Array Biopharm). The synthesis and use of such analogue peptides and the organic mimetics thereof in order to inhibit intracellular signalling are known to a person skilled in the art (see for example EP2060265 B1).

The use of a TrkA inhibitor only, in the treatment of cancer, has only a limited efficacy in that the independent signalling paths for phosphorylation of TrkA are also involved in the invasion of cancer cells. The additional use of an EphA2 inhibitor thus makes it possible to inhibit an independent signalling path for phosphorylation of TrkA.

The ephrin type-A receptor 2 (EphA2) is a 135 kDa receptor with tyrosine kinase activity. The group A ephrins are bound to the source cell membrane by a glycosyl-phospatidylinositol, whereas the group B ephrins have a transmembrane domain.

“EphA2 inhibitor” shall mean an EphA2 antagonist, an inhibitor of the expression of the EphA2 receptor, or a molecule preventing the binding of signalling adapter molecules. Preferably, an EphA2 inhibitor refers to an EphA2 antagonist or an inhibitor of the expression of the EphA2 receptor, more preferably an EphA2 inhibitor refers to an inhibitor of the expression of the EphA2 receptor. In the framework of the present invention, an inhibitor of the tyrosine kinase activity of EphA2 is not considered to be an “EphA2 inhibitor”.

According to a particular embodiment, said inhibitor according to the invention is an EphA2 antagonist. An antagonist is typically any substance, simple or complex compound, of natural or synthetic origin which opposes the activation of the EphA2 receptor. In general, such an antagonist alters the signalling or recruitment of EphA2 partners in the cell.

For example, the EphA2 antagonist is an anti-EphA2 antibody which neutralizes EphA2 or an anti-EphA2 fragment of same which neutralizes EphA2.

Anti-EphA2 antibodies are known to a person skilled in the art. Reference can be made for example to application WO2009028639, application WO0112840 or application WO2008010101.

Antibodies against EphA2 can also be produced according to methods known to a person skilled in the art. It is thus possible to refer to the preceding paragraphs mentioning such production methods which are also applicable here.

Although the antibody according to the invention can be a polyclonal antibody, a monoclonal antibody is preferred. Monoclonal antibodies against EphA2 can be produced and isolated according to methods known to a person skilled in the art. It is also possible here to refer to the preceding paragraphs mentioning such production methods which are also applicable here.

Antagonists that can be used according to the invention also include antibody fragments, for example but not limited to, Fab, F(ab')₂ and scFv fragments.

Humanized anti-EphA2 antibodies or antibody fragments can also be prepared according to techniques known to a person skilled in the art. It is also possible here to refer to the preceding paragraphs mentioning such production methods which are also applicable here.

For this invention, the neutralizing antibodies of EphA2 are selected.

According to another example, the EphA2 antagonist is selected from the aptamers. The aptamers can be produced according to methods known to a person skilled in the art. It is thus possible to refer to the preceding paragraphs mentioning such production methods.

For this invention, the neutralizing aptamers of EphA2 are selected.

According to another example, the EphA2 antagonist is a natural ligand of EphA2 having been modified in such a way as to prevent the binding of a natural EphA2 ligand. Ephrin A1 is thus a natural ligand of EphA2. Other natural ligands, with lower affinities, are the members of the ephrin A family. A person skilled in the art knows how to modify the natural ligands of the receptor in such a way as to produce antagonist molecules. For example, it is possible to use a natural ligand or a natural ligand fragment, preferably of ephrin A1, coupled to the Fc part of an antibody (also known as ephrin A1-Fc) (55).

According to a particular preferred embodiment, the inhibitor according to the invention is an inhibitor of the expression of the EphA2 receptor (i.e. of the EphA2 receptor gene).

Small inhibitory RNA (siRNA) can be used according to the invention as EphA2 receptor expression gene inhibitors. The expression of the gene for the EphA2 receptor can be reduced by bringing a subject or a cell into contact with small fragments of double-stranded RNA (dsRNA), or a vector or a genetic construction inducing the production of small fragments of double-stranded RNA, in such a way that the expression of the EphA2 receptor gene is specifically inhibited (i.e. RNA interference or RNAi). Methods for selecting a dsRNA or a vector coding for dsRNA are known in the prior art. It is possible to refer to the preceding paragraphs alluding to such production methods. Reference can also be made to the publication by Haifa Shen et al. (25).

Ribozymes can also be used according to the invention as inhibitors of the expression of the EphA2 receptor gene.

The antisense siRNA oligonucleotides and the ribozymes able to be used as inhibitors for the expression of the EphA2 receptor gene can be prepared and selected according to known methods. Reference can be made to the preceding paragraphs which mention such methods that are also applicable here.

Finally, it is possible to use a molecule preventing the binding of signalling adapter molecules as an EphA2 inhibitor. “Signalling adapter molecule” shall mean molecules which interact with the receptors via the interaction domains in order to trigger intracellular signals. The signalling adapter molecules of the EphA2 receptor are for example Shc, Fak-1, ephexin-4. These inhibitors can be peptides for example, generally obtained by synthesis of all or part of a signalling molecule, which would have a negative dominant effect on the bonds of cellular adapters. These molecules can also be organic mimetics, i.e. non-peptide analogues, the conformation of which enables recognition of the EphA2 receptor sequence in order to bind there and prevent by competition the binding of the signalling partner. Through their bonding, these inhibitors can also prevent activation of the EphA2 receptor by inhibiting the changes in conformation necessary for EphA2 activation (for example, by an allosteric antagonist such as ALW-II-41-27 (26)). The synthesis and use of such analogue peptides and the organic mimetics thereof in order to inhibit intracellular signalling are known to a person skilled in the art (see for example EP2060265 B1).

It is generally known that, in the normal epithelial cells of the breast, EphA2 is expressed at the cell surface and bonds to its ligand, ephrin-A1. The ligand/receptor interaction contributes to tissue homoeostasis. The overexpression of EphA2 has previously been reported as playing a determining role in the invasive and oncogenic capacities of breast cancer cells (27). The EphA2 receptor has been associated with a basal-type phenotype and an aggressive behaviour in tumour cells (27,28).

However, it has been shown by the inventors that proNGF recruits TrkA by binding on sortilin, but also that TrkA associates directly with EphA2. The proNGF therefore causes the direct association of the sortilin/TrkA complex and EphA2. As previously mentioned, the inventors have discovered that the complex associating sortilin, TrkA and formed EphA2, induces independent signalling pathways for phosphorylation of TrkA, such as for example the activation of the protein Src, also intervening in cancer cell invasion.

Contrary to what was previously imagined, the inventors have also shown that Src is not induced by phosphorylation of TrkA but by an independent signalling of this phosphorylation, requiring the recruitment of EphA2 on the sortilin/TrkA complex. The inhibition of TrkA only, does not therefore allow effective inhibiting of said protein, Src. Src is moreover an oncoprotein, the activation of which in cancer cells is often associated with resistance to therapies.

EphA2 inhibition therefore allows, for example, inhibiting of the activation of the protein Src, as well as all of the pro-tumoral signalling induced by EphA2, such as for example signalling dependent on Ras and MAP-kinases (29), Vav2-RhoA (30) and Stat 5 (31).

Thus, a treatment based on the use of a TrkA inhibitor and an EphA2 inhibitor makes it possible to intervene, in a synergistic and effective manner, against cancer by overcoming the resistance phenomena which arise with a treatment based on a TrkA inhibitor only, for example a treatment based on lestaurtinib.

Furthermore, in this case tyrosine kinase activity receptors are activated under the effect of proNGF, i.e. a ligand of tyrosine kinase activity receptors not having been subject to maturation. It has indeed been shown by the inventors that these signalling pathways are specifically activated by proNGF, NGF itself activating proteins Akt and Src in a manner dependent on the phosphorylation of TrkA, without requiring sortilin or EphA2. proNGF therefore does not act in a similar manner to NGF, and induces cancer cell invasion in a very specific manner.

Here “cancer treatment” means any treatment capable for example of eliminating a tumour or metastases, reducing the risk of recurrence, slowing the development of a tumour or metastases, and/or treating the symptoms of the illness.

The cancers targeted by the present invention are in particular those in which TrkA and EphA2 receptors are expressed, and preferably also the association of TrkA and EphA2 receptors.

The detection of cancers targeted by the present invention can be performed according to techniques known to a person skilled in the art. Typically, the TrkA and EphA2 receptors are detected. Preferably, the association of protein complexes, in particular the association of TrkA and EphA2 receptors, and possibly of TrkA and EphA2 receptors and sortilin, is also detected. In the following, the TrkA and/or EphA2 receptor are referred to as the “receptor to be detected”.

For example, but not in a limiting manner, TrkA and EphA2 receptors can be detected in a biological sample by detection of the mRNA of TrkA/EphA2, by direct detection of the TrkA/EphA2 proteins or by demonstrating TrkA/EphA2 activation.

A biological sample used for the direct detection of TrkA/EphA2 can be a biological fluid or a tissue from a tumour or metastases biopsy of the patient.

Biological fluids that can be used according to the present invention include blood, bone marrow, milk, cerebrospinal fluid, urine and effusions.

The biological fluid may require a special treatment, particularly if coming from circulating tumour cells which express the receptor to be detected. Hence, according to an embodiment of the invention, the biological fluid maybe treated beforehand in order to isolate the circulating tumour cells or circulating cell fragments (exosomes or microvesicules) contained in said biological fluid. Here, “isolating circulating tumour cells” shall mean obtaining a cellular fraction that is enriched in circulating tumour cells. The treatment of the fluid in order to isolate circulating tumour cells may be carried out, for example, by cell sorting in a flow cytometer, by enrichment on Ficoll, by enrichment using magnetic beads coated with specific antibodies, or by any other specific enrichment method known to a person skilled in the art. The circulating cell fragments can be prepared using preparative methods such as ultracentrifugation or any other chromatographic methods, for flow cytometry allowing isolation and enrichment known to a person skilled in the art.

In the case where the biological sample is blood or bone marrow, the circulating tumour cells can be isolated using a cellular separation technique on Ficoll in combination with a depletion of the blood cells using anti-CD45 antibodies coupled to magnetic beads (Dynal Biotech ASA, Norway). The direct detection of the receptor can then be carried out directly using the circulating tumour cells isolated from the biological fluid by any means well known to a person skilled in the art, for example by immunocytochemical marking of the cells with an antibody or by flow cytometry.

When the biological sample is a tissue sample, typically coming from a biopsy of the tumour or metastases of the patient, the direct detection of the receptor can be carried out directly on the segments obtained, without prior treatment of said tissue.

The direct detection of the targeted receiver can be implemented by any means known to a person skilled in the art, in particular by mass spectrometry or by immunological tests. The immunological test can be any test widely known by a person skilled in the art involving immunological reactions, i.e. reactions between a receptor and a specific bonding partner of the receptor.

Mass spectrometry can be used for the direct detection of TrkA in the biological sample. The principles and implementation of mass spectrometry are widely known to a person skilled in the art.

For example, the biological sample, optionally treated beforehand (for example by passage on an immunocapture medium, comprising one of the bonding partners of the receptor, for example an antibody), is passed into a mass spectrometer and the obtained spectrum is compared with that of the receptor to be detected (for example, TrkA or EphA2). The detection can also be made by marking the membrane proteins (for example with biotin) for capture by streptavidin/biotin affinity.

Immunological tests are also well known to a person skilled in the art and include immunological reactions, i.e. reactions between the receptor to be detected and a specific bonding partner of the receptor to be detected.

The specific bonding partners of the receptor to be detected are any partner able to bond to the receptor. By way of example, these include antibodies, antibody fractions, receptors and any other protein capable of bonding to the receptor to be detected. The bonding partner antibodies are typically either polyclonal antibodies or monoclonal antibodies. Techniques for obtaining such antibodies are well known to a person skilled in the art. Furthermore, examples of anti-TrkA antibodies are known and are available, in particular in the Cell Signaling or Alomone catalogue. Examples of anti-EphA2 antibodies are also known and available, in particular in the Cell Signaling or Santa Cruz catalogues.

Marking of specific bonding partners of the receptor to be detected can also be carried out to reveal the receptor/bonding partner bonding. Here, “marking of the bonding partners” shall mean binding of a marker capable of directly or indirectly generating a detectable signal. A non-restrictive list of said markers consists of enzymes which produce a detectable signal, for example by colorimetry, fluorescence, luminescence, such as horseradish peroxidase, alkaline phosphate, alpha-galactosidase, glucose-6-phosphate dehydrogenase, chromophores such as fluorescent, luminescent and staining compounds; fluorescent molecules such as alexa or phycocyanins; radioactive molecules containing radioactive isotopes of elements such as P, S or I, etc.

Indirect marking systems can also be used, such as for example ligands capable of reacting with an anti-ligand. Ligand/anti-ligand pairs are well known to a person skilled in the art, for example biotin/streptavidin, hapten/antibodies, antigen/antibodies, peptide/antibodies, sugar/lectin, polynucleotide/polynucleotide complement. The anti-ligand can be directly detectable by the previously described markers or can itself be detectable by a ligand/anti-ligand pair.

Immunological tests such as those defined above include, by way of example, “sandwich” methods such as ELISA, IRMA and RIA, so-called competition methods and direct immunodetection methods such as immunohistochemistry, immunocytochemistry, the western blot test and the dot blot test.

Another method for detecting the presence of the receptor to be detected involves culturing cells sensitive to the receptor to be detected, in the presence of the biological sample, which constitutes a particular embodiment of the invention.

Another particular embodiment for detection of the presence of the receptor to be detected in the biological samples consists of detecting the mRNA of said receptor to be detected inside said sample.

The detection of mRNA, preferably from circulating cells or circulating fragments in a biological fluid as defined above, is widely known to a person skilled in the art. The detection can for example be implemented by hybridization reactions between the target mRNA and a nucleic acid capable of bonding with the target mRNA.

Here nucleic acid shall mean oligonucleotides, deoxyribonucleic acids and ribonucleic acids, and the derivatives thereof. The term “oligonucleotide” refers to a chain of at least two natural or modified nucleotides (deoxyribonucleotides or ribonucleotides, or both), able to be hybridized under appropriate hybridization conditions, with one oligonucleotide being at least partly complementary. Here “modified nucleotide” shall mean, for example, a nucleotide comprising a modified base and/or comprising a modification of the internucleotide bond and/or the backbone. Examples of the modified base include inosine, methyl-5-deoxycytidine, dimethylamino-5-deoxyuridine, diamino-2,6-purine and bromo-5-deoxyuridine. For purposes of illustration only, a modified internucleotide bond may be a phosphorothioate, N-alkylphosphoramidate, alkyl phosphonate and alkylphosphodiester bond. The alpha-oligonucleotides such as those described in FR-A-2 607 507, the LNA such as phosphorothioate-LNA and 2′-thio-LNA described in Bioorganic & Medicinal Chemistry Letters (32), and the PNA (33), are examples of oligonucleotides consisting of nucleotides the backbone of which is modified. The hybridization reactions can be revealed by marking bonding nucleic acids, as illustrated above.

Before hybridization with the bonding nucleic acid, the target mRNA can be extracted by methods known to a person skilled in the art, then optionally amplified, for example by RT-PCR or by NASBA (34).

The detection and association of protein complexes can also be carried out by any means known to a person skilled in the art, in particular by immunological tests. Typically, the proximity ligation assays (PLA) method or Duolink® can be used. The detection of protein complexes can also be implemented by bivalent reagents.

Advantageously, a solid cancer targeted by the present invention, is a solid cancer having cancer cells expressing EphA2 and TrkA receptors, which is selected from breast cancer (35, 36), prostate cancer (37,38), colon cancer (36,37), tongue cancer (36,39), cancer of the oropharyngeal sphere (39,39), thyroid cancer (40,41), pancreatic cancer (36,42), neuroblastoma (36,43), glioma (44) and skin cancer (melanoma) (36,45). Preferably, a cancer targeted by the present invention is breast cancer, prostate cancer or cancer of the oropharyngeal sphere (tumours of the tongue, buccal cavity, pharynx and larynx). More preferably, a cancer targeted by the present invention is breast cancer.

The subject matter of the invention relates to a method for treating solid cancer in a patient treated by a TrkA inhibitor as described above, comprising a step of administering to said patient a therapeutically active quantity, that is tolerable from a pharmacological point of view, of an EphA2 inhibitor such as described above.

A subject of the invention also relates to a method for treating solid cancer in a patient treated by an EphA2 inhibitor as described above, comprising a step of administering to said patient a therapeutically active quantity of a TrkA inhibitor such as described above.

Here the term “patient” shall mean a human being affected, or likely to be affected, by a solid cancer targeted by the present invention, as defined above.

The term “therapeutically active quantity” of an inhibitor according to the invention means a sufficient quantity of inhibitor for treating such a cancer, having an acceptable risk-benefit ratio for a medical treatment. The quantity and composition of inhibitor(s) according to the present invention, as well as the frequency of administration, will be determinable by clinical studies performed by a doctor or pharmacist. The “therapeutically active” quantity specific to each patient will depend on a certain number of factors, such as the nature and severity of the disorder to be treated, the inhibitor used, the composition used, the age, weight, general state of health, sex and diet of the patient, the method of administration, the duration of the treatment (in a single dose or multiple doses), the drugs used in combination and other factors well known to medical specialists.

Typically, the TrkA inhibitor according to the invention and the EphA2 inhibitor according to the invention can be administered simultaneously or sequentially.

Advantageously, the TrkA inhibitor according to the invention and the EphA2 inhibitor according to the invention can be combined in a pharmaceutical composition or can be separate, for example in the form of a combination product (kit).

A subject of the invention also relates to a pharmaceutical composition comprising a TrkA inhibitor such as described above, an EphA2 inhibitor such as described above, and at least one pharmaceutically acceptable carrier.

A particular embodiment relates to such a pharmaceutical composition for use in the treatment of solid cancer.

In the pharmaceutical composition according to the invention, the TrkA inhibitor and the EphA2 inhibitor are combined, in therapeutically active quantities, with one or more pharmaceutically acceptable carriers, and optionally with extended release matrices, such as a biodegradable polymers, in order to form the therapeutic compositions.

Here “pharmaceutical” or “pharmaceutically acceptable” shall mean any molecular entity and composition which does not produce negative allergic reactions, or any other desired reaction during administration to a mammal, in particular a human being. A pharmaceutically acceptable carrier or excipient refers to a solid, semi-solid, diluent, encapsulation material, or any other non-toxic formulation. For example, sterile saline solutions buffered with phosphate are pharmaceutically acceptable. Pharmaceutically acceptable carriers can usually include one or more compounds, selected for example from excipients, preservatives, solubilizing agents, buffer agents, albumin etc. Known excipients are for example starch, gelatin, stearic acid, calcium stearate or magnesium stearate, etc.

The form of the pharmaceutical composition, the mode of administration, the dosage and posology can depend, inter alia, on the cancer to be treated, the symptoms thereof, the severity thereof, the age, weight and/or sex of the patient.

In a non-restrictive manner, the pharmaceutical composition according to the invention can be formulated in such a way as to be administered by the oral, sublingual, subcutaneous, intramuscular, intratumoural, intravenous, topical, local, intratracheal, intranasal, transdermal, rectal, intraocular or intra-auricular routes; the inhibitors according to the invention can be administered, individually or in combination, in the form of a single administration.

The single administration forms can be for example, tablets, capsules, granules, powders, orally injectable solutions or suspensions, transdermal patches, with sublingual, buccal, intratracheal, ocular, intranasal, intra-auricular forms of administration, or by inhalation or with transdermal, subcutaneous, intramuscular, intratumoural or intravenous forms of administration, or with rectal forms of administration or implants. Creams, gels, ointments, lotions or eye-drops can be envisaged for topical administration.

These galenic forms are prepared according to the usual methods of the domains considered.

A further subject of the invention relates to a combination product comprising:
a TrkA inhibitor such as described above, and
an EphA2 inhibitor such as described above.

A particular embodiment relates to such a combination product for use in the treatment of solid cancer.

Such a combination product or kit enables administration of TrkA inhibitors and EphA2 inhibitors in accordance with the invention in a simultaneous manner, i.e. at the same time, or at separate times, i.e. successively.

The invention also relates to a method for prognosis of the survival of a patient suffering from a solid cancer, comprising a step of detecting the expression of TrkA and EphA2 in a biological sample of the patient, the co-expression of TrkA and EphA2 being associated with a poor prognosis of survival of the patient.

Typically, the step of detecting the expression of TrkA and EphA2 is carried out by detecting a TrkA/EphA2 complex.

Indeed, the co-expression of TrkA and EphA2, in particular in the presence of a TrkA/EphA2 complex, is associated with a significant reduction in the overall survival of patients.

A biological sample used for the direct detection of TrkA/EphA2 is typically a sample from a tumour or metastases biopsy of the patient.

The detection of the co-expression of TrkA and EphA2 can be carried out by any means known to a person skilled in the art.

The detection of the TrkA/EphA2 complex can be carried out by any means known to a person skilled in the art, in particular by immunological tests. Typically, the proximity ligation assay method (PLA) or Duolink® can be used. The detection of the complex can also be implemented by bivalent reactions which recognize both TrkA and EphA2.

### FIGURES

FIG. 1: A study of the pro-invasive effects of NGF and proNGF on cancerous epithelial cells. The invasion tests were carried out on cancer cells from the tongue (CAL 27, CAL 33), larynx (SQ20B), pharynx (FaDu), prostate (DU145, PC3) and breast (MDA-MB-231), seeded in a Boyden chamber and treated with NGF (16 nM) or proNGF N.C. (0.5 nM) for a period of 20 hours. The untreated cells represent the control and determine a base invasion of 100%.

FIG. 2: A study of the expression of receptors in cancerous epithelial cells. The levels of protein expression of the EphA2 receptors, sortilin, p75NTR, and TrkA have been measured by the western blot method on cells sensitive to proNGF (cells CAL 33, DU145, PC3, and MDA-MB-231). A total of 50 μg of proteins were loaded.

FIG. 3: A study of the involvement of EphA2 on the pro-invasive effects of proNGF. The invasion test is carried out on cells MDA-MB-231 (A), DU145 (B) and PC3 (C) transfected with a siRNA against EphA2 (siEphA2), TrkA (siTrkA) or a random siRNA (siCTRL). The transfected siCTRL cells are treated to provide a control and determine a base invasion of 100% (white column). For statistics, the error bars represent the standard deviation. * p<0.001 for proNGF or NGF treatment vs. no treatment; §p<0.001 for the experimental vs. the control with treatment of proNGF. The efficacy of the siRNA is measured by the western blot method through the use of specific antibodies against TrkA, EphA2, and actin (load control).

FIG. 4:

A—Correlation of the expression of sortilin (SORT1), TrkA (NTRK1) and EphA2 with metastasis free survival by microarray analysis (SORT1), EphA2 and TrkA (NTRK1) after over-regulation of the sortilin, TrkA and EphA2 (lower curve) or without over-regulation (upper curve).

B—Expression of the TrkA/EphA2 complex is associated with a reduction in the overall survival of patients in breast cancer. The expression of the TrkA/EphA2 complex was investigated by proximity ligation assays on 182 breast tumours (tissue microarrays CBA4 (Superbiochips) and Hbre-Duc 150Sur-01 (US Biomax)). The expression of the complex has been graded as zero (no marking) weak or very weak expression (0 to 5 complexes detected per cell on average; medium 5 to 15 complexes per cell on average; strong more than 15 complexes per cell on average). The overall survival is reported in the form of a Kaplan Meier graph; the significance is obtained using a log-rank test (n=182, p<0.0001).

FIG. 5: Identification of the proteins associated with TrkA in mass spectrometry.

FIG. 6: A study of the formation of a complex between TrkA, EphA2 and sortilin by immunoprecipitation in cells MDA-MB-231 HA-TrkA. The cell MDA-MB-231 HA-TrkA were treated with 0.5 nM of proNGF N.C or 16 nM of NGF (5 and 30 min). HA TrkA, sortilin and EphA2 were immunoblotted by using, respectively, the antibodies anti-HA, anti-sortilin and anti-EphA2.

FIG. 7: A study of the impact of a sequential invalidation of each of the receptors TrkA, sortilin and EphA2 on the formation of the TrkA/sortilin/EphA2 complex, during treatment by proNGF.

A. Cells MDA-MB-231 HA-TrkA were transfected with siTrkA and then treated with proNGF N.C. The sortilin was immunoprecipitated, EphA2 and HA-TrkA were immunoblotted.

B. Cells MDA-MB-231 HA-TrkA or kinase-dead HA-TrkA were treated with proNGF N.C. The recruitment of the sortilin and EphA2 was determined after anti-HA immunoprecipitation. The efficacy of the mutagenesis has been verified with an anti-phosphotyrosine antibody.

C. Cells MDA-MB-231 HA-TrkA were treated with proNGF N.C in the presence or absence of 1 μM neurotensin. HA-TrkA was immunoprecipitated with anti-HA antibody, and sortilin and EphA2 were immunoblotted.

D. Cells MDA-MB-231 HA-TrkA were transfected with siEphA2 and then treated with proNGF N.C. (30 min). The recruitment of the sortilin and EphA2 was determined after anti-HA immunoprecipitation.

FIG. 8: A study of the role of TrkA and EphA2 in the activation of proteins Akt and Src. Cells MDA-MB-231 were respectively treated with a TrkA inhibitor (K252a), an anti-EphA2 siRNA (siEphA2), an inhibitor of PI3-K (LY294002) and an inhibitor of Src (SKI-1).

A and D. Cells MDA-MB-231 HA-TrkA were treated with proNGF N.C. (A) or NGF (D) (30 min) in the presence or absence of 10 nM K252a, 15 μM LY294002 and 50 nM SKI-1. The phosphorylation of Akt and Src was determined by using phospho-specific antibodies. The equal charge was verified using Akt and Src antibodies.

B and E. Cells MDA-MB-231 HA-TrkA or kinase-dead HA-TrkA were treated with proNGF N.C. (B) or NGF (E) (5 and 30 min). The phosphorylation of Akt and Src was measured by the western blot method.

C and F. The phosphorylation of Akt and Src was determined in the total lysates coming from cells MDA-MB-231 transfected with siEphA2 and treated for 30 minutes with proNGF N.C. (C) or NGF (F).

FIG. 9: A study of the inhibition of TrkA and EphA2 on tumour development in vivo. The tumours were developed without any intervention for 14 days and then subjected to 3 injections (every 3 days, black arrows) of solvent (control), CEP-701 (10 mg/kg), siEphA2 (7.5 microg/mouse) or CEP-701 (10 mg/kg) and siEphA2 (7.5 microg/mouse). The experiments were stopped 31 days after the first injection of the treatment for ethical considerations after the death of the control mice. A) The tumour volumes were evaluated by measuring the length (;) and the width (w) and then calculating the volume using the formula π/6×l×W×(l+w)/2; the Mann and Whitney test was carried out between the control group and CEP-701 (a), between the control groups and siEphA2 (b), between the control groups and CEP-701+siEphA2 (c), and between the groups siEphA2 and CEP-701+siEphA2 (d)*, P<0.05; **, P<0.01; ns: not significant. B) Study of the inhibition of the expression of receptors by siRNA on the overall survival of SCID mice. The tumours were developed without any intervention for 14 days and were then subjected to 5 injections (D14, DI7, D20, D23, D26) of SiTrkA (7.5 microg/mouse), siEphA2 (7.5 microg/mouse), siTrkA (7.5 microg/mouse) and siEphA2 (7.5 microg/mouse). For ethical considerations, the limit points were defined by a tumour volume not exceeding 2.5 cm³ and a maximum duration of 90 days after inoculation of the tumour cells. Beyond these limit points, the mice were euthanized. The tumour volumes were evaluated by measuring the length (l) and width (w) and then calculating the volume using the formula π/6×l×W×(l+w)/2. Survival is represented in the form of a Kaplan Meier graph. Significance was measured using the log-rank test (P=0.0022). The median survival was respectively 40 days for the control, 53 days in the presence of siTrkA, 49 days in the presence of siEphA2 and 57.5 days in the presence of siTrkA and siEphA2.

FIG. 10: Study on the effect of lestaurtinib (TrkA inhibitor) and EphrinA1-Fc (EphA2 inhibitor) and combination of treatment on invasion of breast cancer cells. The invasion test was carried out on the cells MDA-MB-231. Non-stimulated cells (“non stim”) of proNGF served as the control and determined a base invasion of 100% (white bar).

### EXAMPLES

### Example 1

EphA2 was involved in the cellular invasion of various lines of cancerous cells induced by proNGF through a functional interaction with the sortilin/TrkA complex.

Prior research had demonstrated that numerous epithelial cancer cells responded to NGF or to proNGF (24, 25, 51-54). The effects of NGF and proNGF on the invasion capacity of various cancer lines was therefore tested.

The experiments were carried out on cancer cells from the tongue (CAL27, CAL33), larynx (SQ20B), pharynx (FaDu), prostate (DU145, PC3) and breast (MDA-MB-231).

### Chemicals and Reagents

The reagents were supplied by Sigma (France) and the cell culture media were obtained from Invitrogen (France). Cell culture consumables and plastics came from BD-Falcon (France) and Greiner (France). The recombinant human β NGF was supplied by Scil Proteins (Germany), the recombinant human non-cleavable proNGF (proNGF N.C.) by Alomone Labs (Israel) and the pharmacological inhibitor K252a was obtained from Calbiochem (United Kingdom). The antibodies came from Cell Signaling Technology (France) except for the anti-HA antibodies (Covance; France) and anti-sortilin (R&D Systems; France).

### Cell Cultures

The cell lines CAL 27, CAL 33, SQ20B and FaDu (cancer lines from the upper aerodigestive tracts) were supplied by the Centre Oscar Lambret (Lille), and the cell lines DU145 and PC3 (prostate cancer lines), and MDA-MB-231 (breast cancer lines) came from the American Tissue Culture Collection (ATCC, Manassas, Va., USA).

Cells were maintained in minimum essential medium from Eagle (MDA-MB-231), medium from Dulbecco (CAL 27, CAL 33 and SQ20B), medium RPMI 1640 (DU145, PC3 and FaDu) containing 10% inactivated foetal calf serum (FCS) (Hyclone, France), 2 mM L-glutamine, 1% non-essential amino acids, 40 UI/ml penicillin, 40 μg/ml streptomycin, 50 μg/ml gentamicin and in the presence of ZellShield™ (IX, Biovalley, France) (37° C., 5% CO₂ in a saturated humidity atmosphere).

The breast cancer line (MDA-MB-231) overexpressing HA-TrkA (hereinafter named “MDA-MB-231 HA-TrkA”) was established in the laboratory by stable overexpression of the TrkA receptor (variant 1 NM_001012331.1) which exhibited a silent mutation at position 799, CAA gives CAG (Thr→Gln), and a sequence conflict at position 263 (Val>Leu) (indexed in Swiss Prot) with an HA label (Hemagglutinin: Tyr Pro Tyr Asp Val Pro Asp Tyr Ala) in the N-terminus position.

### Sequential Invalidation Test

The sequences of siRNA used against EphA2 (siEphA2) at 100 pmol for each transfection, and against TrkA (pool of 3 siTrkA 1, 2 and 3) are given in table 1. The transfections of siRNA are produced by means of INTERFERin™ according to the manufacturers instructions (POL409-10, Polyplus transfection, Ozyme, Saint Quentin en Yvelines, France).

TABLE 1

| RNA | |
|---|---|
| inter- | |
| ference | Sequence |
| siEphA2 | GCAAGGAAGUGGUACUGCUGGACUU (SEQ ID NO: 1) |
| siTrkA1 | GAACCUGACUGAGCUCUAC (SEQ ID NO: 2) |
| siTrkA2 | UGGAGUCUCUCUCCUGGAA (SEQ ID NO: 3) |
| siTrkA3 | GCUGCAGUGUCAUGGGCAA (SEQ ID NO: 4) |

### Western Blot

After treatment, the cells were lysed (lysis buffer: 40 mM HEPES pH 7.5; 1 mM EDTA pH 8.0; 120 mM NaCl; 10 mM NaPPi; 50 mM NaF; 1.5 mM Na3VO4; 1% Triton X 100; 0.1% SDS; 1 mM PMSF; 10% glycerol and 1/100 th of protease inhibitor cocktail (Sigma) (20 min; 4° C.)). The lysates were recovered and clarified by centrifugation (12,000 g; 10 min; 4° C.). The supernatant was then stored at −80° C. until use. The protein extracts were diluted to 2 μg·μL−1 in a Laemmli 1× buffer (7 min; 100° C.) then deposited and separated on SDS-PAGE (concentration gel 3.6% acrylamide and separation gel 7.5% to 10% acrylamide (thickness 1 mm); 180 V; 10 min then 200 V constant; until output of the migration front). The proteins separated by SDS-PAGE were transferred according to the semi-dry transfer method (buffer: 48 mM Tris base, 39 mM glycine, 1.64 mM SDS, 20% methanol (v/v); 14 V; 15 min per gel) or liquid (buffer: 25 mM Tris base and 192 mM glycine; 15% methanol (v/v); 100 V; 30 min) on nitrocellulose membranes (0.45 μm; Whatman; Germany) or PVDF (0.45 μm; Immobilon P; Millipore; France). The PVDF membrane was “activated” beforehand (methanol; 10 min; 20° C.). The membranes were then saturated (TBS-T; 5% BSA; 1 h; 20° C.) then incubated (16 h; 4° C.) with the chosen primary antibody. The membranes were then washed (TBS-T), incubated (1 h; 20° C.) with a secondary antibody (diluted to 1/5000 th (anti-rabbit) or to 1/10000 th (anti-mouse) in 5% bovine albumin serum) coupled with HRP (horseradish peroxidase), then revealed using the SuperSignal® West Pico kit (Pierce). The membranes were placed in an image analyser (LAS 4000; Fuji) connected to a computer which allowed image generation (software Image Reader LAS 4000).

The antibody against EphA2 was sc-924 (Santa Cruz), the antibody against sortilin was BD Bioscience 612101, the antibody against TrkA was sc 14024 (Santa Cruz), the antibody against HA was MMS-101R (Covance), the antibody against Akt was Cell Signaling 4691, the antibody against phospho-Akt (Ser 473) was Cell Signaling 9271, the antibody against Src was Cell Signaling 2109 (WB), the antibody against PhosphoSrc (Tyr416) was Cell Signaling 2105 and the antibody against actin was Sigma A2066.

### Immunoprecipitation

The beads (Protein G-agarose, Millipore) were washed twice (12,000 g; 1 min; 4° C.) and recovered in PBS IX (at 50% (v/v)). The protein samples (1 mg) were pre-clarified (1 h; 4° C.; on a wheel (10 rpm)) with 100 μl of beads and 5 μg of isotypical antibodies (mouse IgG2b), centrifuged (12,000 g; 2 min; 4° C.), then the supernatant was incubated (2 h; 4° C.; on a wheel (10 rpm)) with 5 μg of α-HA 12CA5 antibody (Roche). The beads (100 μL at 50% in PBS IX) were added directly to the antibody/protein complex then incubated (2 h; 4° C.; on a wheel (10 rpm)). The antibody/antigen/bead complexes were then centrifuged (12,000 g; 5 min; 4° C.), washed three times (lysis buffer; 4° C.), then disassociated by addition of Laemmli 2× buffer (25 mM Tris-HCl, 0.8% SDS, 4% glycerol, 1% β-mercaptoethanol, pH 6.8) (7 min; 100° C.). Finally, after centrifugation (12,000 g; 10 min; 4° C.), the supernatant was stored at −20° C. The antibody against EphA2 was sc-924 (Santa Cruz), the antibody against sortilin was BD Bioscience 612101, and the antibody against HA was Roche 11 666 606 001.

In situ PLA (Proximity Ligation Assay) in cells MDA-MB-231 HA-TrkA, MDA-MB-231, DU145, PC3 and tissue microarrays.

The cells (20,000 cells per well) were seeded in the Labtek® which had been treated beforehand with alcohol/hydrochloric acid (absolute ethanol+2% HCl). The cells with then weaned (0.1% FCS; 1 h; 37° C.) then treated or not with proNGF N.C. (0.5 nM) or NGF (16 nM) for 5 minutes. After a fixing step (paraformaldehyde (PFA) 4% in PBS 1× (Phosphate Buffered Saline: 2.7 mM KCl, 8 mM Na2HPO4, 1.8 mM KH2PO4, 137 mM NaCl, pH 7.4; 30 min; 20° C.), the cells were incubated with a blocking solution (TBS-T: 20 mM Tris base, 137 mM NaCl, 0.1% Tween 20, pH 7.6, 4% de bovine serum albumin (BSA); 1 h; 20° C. then with the anti-HA, anti-sortilin and anti-EphA2 primary antibodies diluted to 1/50 th (anti-sortilin and anti-HA) or to 1/100 th (anti-EphA2) in the blocking solution. The cells were then washed twice with PBS 1× for 5 minutes then incubated for 2 hours at 37° C. with the anti-mouse or anti-rabbit “plus” and anti-rabbit or anti-goat “minus” marked oligo PLA probe (Olink Bioscience) diluted to ⅕ th in a solution of 4% BSA. The choice of PLA probes depended on the primary antibodies used. After two washings of 5 minutes with TBS-T, the signal was detected by the 613 Duolink® detection kit (Olink Bioscience) according to the manufacturer's instructions. In order to visualize the nuclei, the cells were also incubated with Hoechst 33 258 (1 mM in PBS 1×) then the samples were mounted on a slide using fluorescence mounting medium (Dako). The PLA images (red fluorescence points) were obtained using a fluorescence microscope (immersion lens 100×, λ-excitation: 562 nm, λ-emission: 624 nm, Eclipse Ti microscope; Nikon, France) then analysed using the NIS-Elements BR software from Nikon. A red fluorescence point reflects an interaction between two proteins (separation of less than 40 nm; quantitative method).

The marking of the micro-array tissues was carried out using the PLA Brightfield kit (SigmaAldrich) according to the manufacturer's recommendations. The images were acquired using the Eclipse TiU microscope, Nikon (lens 100×).

The antibody against EphA2 was AF-3035 (R&D Systems), the antibody against sortilin was AF 3154 (R&D Systems), the antibody against TrkA was Alomone labs ANT-018.

### In Vitro Invasion Test

The Boyden Chambers (Transwell®; BD Biosciences; France) were recovered from a biological matrix (type 1 collagen from rats diluted to 400 μg·mL-1 in EMEM-0.1% FCS medium; Millipore). The cells were then seeded with 100,000 cells/well on PET membranes (polyethylene terephtalate; dia.: 10.5 mm; porosity: 8 μm) of Boyden Chambers and held in an EMEM-0.1% FCS medium. The cells were treated with K252a (10 nM) in the upper part of the Transwell®, with or without NGF (16 nM) or proNGF N.C. (0.5 nM) in the lower part. After 20 hours of culture, they were washed twice with PBS 1× and fixed (ice cold methanol; 15 min; −20° C.). The non-invasive cells (upper face of the Transwell®) were eliminated by scraping. The number of cells was estimated after staining with Hoechst 33 258 (1 mM; 30 min; 20° C.) by counting the nuclei thereof using fluorescence microscopy on a total of 5 five randomly chosen fields (lens 10×; λ-excitation: 345 nm; λ-emission: 478 nm; Eclipse Ti) using the ImageJ software. Each experiment was performed in triplicate. Statistical analysis of the results was performed by analysis of variance ANOVA (Bonferroni post-hoc test).

Staining of the Gels with Colloidal Coomassie Blue

After migration, the gels were fixed (16 h, 20° C.) (25% ethanol (v/v) and 2% H3PO4 at 85% (v/v)), washed three times (2% H3PO4 at 85% (v/v); 20 min, 20° C.) and pre-soaked in a 2% solution of H3PO4 at 85% (v/v), 1.1 M (NH4)2SO4, 17% ethanol (30 min, 20° C.). They were then stained in the same solution supplemented with 0.05 g of Coomassie blue G250 (48 h, 20° C., under agitation).

### Destaining of the Gels and Trypsin Digestion

The spots of interest were excised, rinsed with ultra-pure water and destained by successive baths of an acetonitrile solution (ACN)/NH4HCO3 at 50 mM (50/50, v/v, 20° C., under agitation). After total destaining, the spots were dehydrated (ACN 100%, 3×10 min, 20° C.) then rehydrated (NH4HCO3 100 mM, 10 min, 20° C.). They were then reduced (45 min, 56° C. with 10 mM DTT in 100 mM NH4HCO3) and alkylated (30 min, 20° C., to obscurity with 55 mM iodoacetamide in 100 mM NH4HCO3). After drying with Speed-Vac (30 min, 20° C.), the spots were rehydrated and incubated with trypsin (NH4HCO3 25 mM, 12.5 μg/ml of trypsin (Promega; Charbonnières les bains, France), 1 h, 4° C.). This solution was then renewed, without trypsin and the tubes were incubated (12 h, 37° C.). After recovering the supernatant, the pieces of gel were cleaned successively in a solution of ACN/formic acid (45/10, v/v) (two washings) then in a solution of ACN/formic acid (95/5, v/v). The supernatants were collected, grouped together with the previous, and all evaporated with the Speed-Vac.

### Analysis by MS/MS Mass Spectrometry

The nanoLC-nanoESl-MS/MS analyses were carried out on an ion trap (LCQ Deca XP+, Thermo Electron; Brebières, France) equipped with a nano-electrospray source coupled to a nano-high-performance liquid chromatography (LC Packings Dionex; Voisins le Bretonneux, France). The tryptic digestates were recovered in 4 μl of a solution with 0.1% formic acid and 1.4 μl was injected by using a Famos auto sampler (LC Packings Dionex). The samples were initially desalinated then concentrated on an inverse phase C18 pre-column (length 5 mm and internal dia. 0.3 mm, LC Packings Dionex) by a solvent A (H2O/ACN, 95/5, v/v; 0.1% formic acid), delivered by the Switchos® pumping system (LC Packings Dionex) at a flow rate of 10 μl/min for 3 minutes. The peptides were separated on a C18 Pepmap column (15 cm×75 μm internal dia., LC Packings Dionex). A constant flow rate was applied (200 nL/min). Les peptides were eluted for 45 min using a linear gradient of 5 to 70% of a solvent B (H2O/ACN, 20/80, v/v; 0.08% formic acid). A voltage of 1.5 kV was applied to the nano-electrospray needle (external dia.: 360 μm, internal dia.: 20 μm, internal dia. of the needle point: 10 μm, covered with a conductive alloy, New Objective, Wil, Switzerland). The analyses were carried out in positive mode. The data acquisition was performed in automatic peptide sequencing mode which consists of alternating an MS spectrum between m/z 500-2000 and an MS/MS spectrum of the most intense ion from the previous MS spectrum. The MS/MS spectra were acquired with an isolation window of the parent ion of 2 uma and with a collision energy of 35%. The MS/MS.raw files were transformed into .dta files using the Bioworks 3.1 software (Thermo Electron). The .dta files were then compiled using the merge.bat software downloadable via the Mascot Daemon software version 2.1.6 (www.matrixscience.com) in order to interrogate the databases on SwissProt 51.4 (252616 sequences). The search parameters were as follows: Homo sapiens (taxonomy), an authorized site “missed cleavage” by the trypsin, carbamidomethylation, oxidation of methionines and phosphorylation of the Ser, Thr and Tyr residues (variable modifications), 2 Da (peptide mass tolerance) and 0.8 Da (MS/MS mass tolerance).

### Analysis of the Expression of Genes in Breast Tumour Samples.

The expressions of sortilin (SORT1), EphA2 and TrkA (NTRK1) were analysed on samples of tumours and breasts. The analysis was carried out on the basis of data collected from 35 cohorts published on the site of the National Center for Biotechnology Information (NCBI)/Genbank GEO database, from the European Bioinformatics Institute (EBI) ArrayExpress database and from the Institut Paoli Calmettes (Marseilles). 6183 cases of non-invasive breast cancer were analysed for the expression of NTRK1 and correlated with clinicopathological data. Pre-analytical processing of the data was then applied. The datasets coming from Agilent DNA chips were normalized by the quantiles to be applied in order to obtain the processed data. For the data generated by the Affimetrix DNA chips, the RMA normalization method (Robust Multichip Average) (48) was used with the algorithm for non-parametric calculating of the quantiles. In order to make all of the data comparable and to exclude any bias arising from the heterogeneity of the populations, the level of NTRK1 expression was standardized inside each data set by taking the luminal A population as the reference, the molecular subtype of the tumours being defined by the PAM50 “Predictor” (49). When several probes were studied with NTRK1, those with the highest variants in a particular dataset were selected. The over-regulation of NTRK1 was defined by the increase in expression above the median level. The correlations between NTRK1 expression and the histochemical variables include the age of the patients at the time of diagnosis (≦50 years vs. >50), pathological state of the axillary lymph node (pN: negative vs. positive), the size of the tumour (pT: pT1 vs. pT2-3), the stage (I vs. 2-3), immunocytochemistry (IHC) of the estrogen receptor alpha (ER), of the progesterone receptor (PR), the status of the ERBB2 receptor (positive vs. negative) for each patient, and the duration of metastases free survival (MFS) for patients not exhibiting metastasis at the time of diagnosis, were investigated. The metastases free survival was calculated from the date of diagnosis to the date of the first metastases discovered. The survival of the patient was measured from the date of diagnosis to the date of first recurrence (follow-up). Survival is calculated by the Kaplan Meier method and the curves are analysed by a log-rank test. Distributions were analysed by Fisher test. Significance was considered with a threshold of 5%. All the analyses were carried out following the “REcommendations for tumor MARKer prognostic studies” (REMARK criteria) (50).

### RESULTS

### Cell Invasion Tests

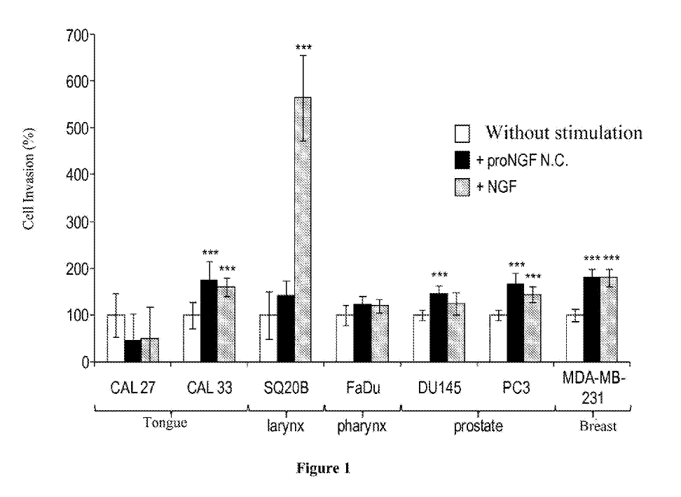

After 24 hours of treatment, it was observed that NGF and proNGF increased cell invasion for cancer lines CAL33, DU145, PC3 and MDA-MB-231. In contrast, tumour cells FaDu and CAL27 did not respond to any growth factors, while the cell invasion of SQ20B was induced uniquely during treatment with NGF (FIG. 1).

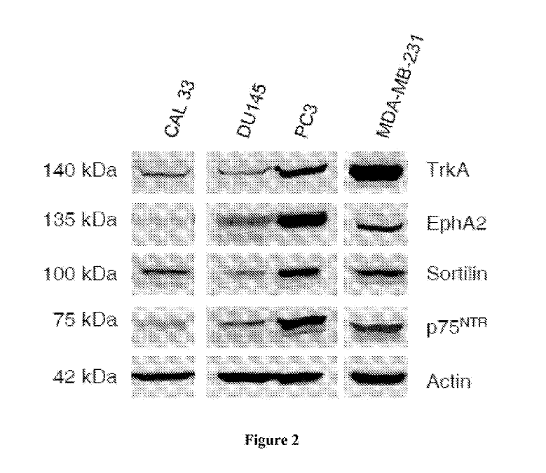

Analyses using the Western Blot method were then carried out in order to evaluate the levels of EhA2, sortilin and TrkA in the cells responding to proNGF (CAL33, DU145, PC3 and MDA-MB-231) (FIG. 2). These three receptors are expressed in each of the tested cell lines, however CAL33 only very weakly expressed EphA2.

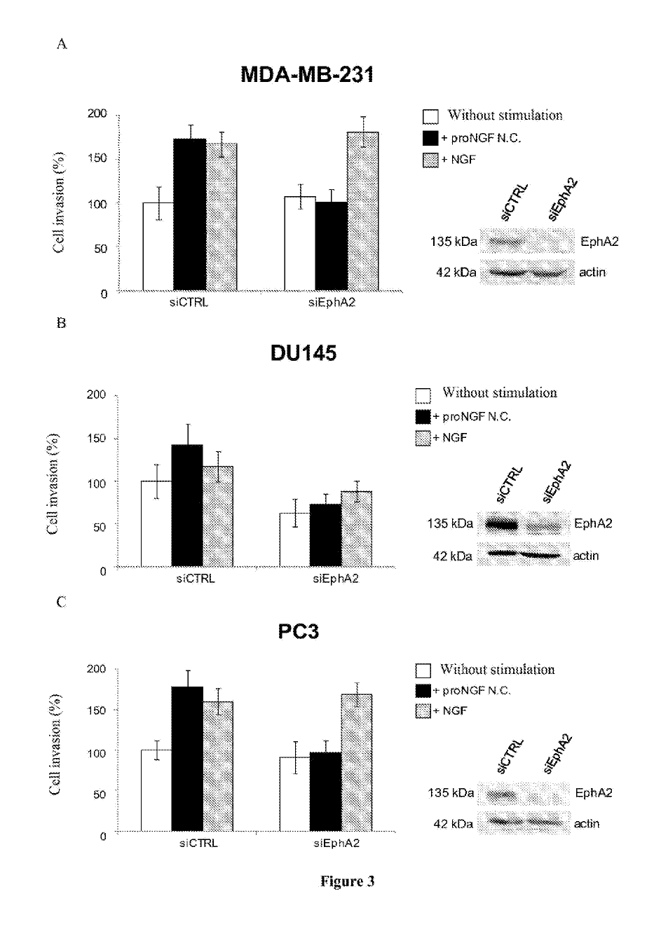

The involvement of EphA2 in the pro-invasive effects of proNGF was evaluated using a Boyden chamber on the cells overexpressing EphA2 (DU145, PC3 and MDA-MB-231). As shown in FIG. 3, siEphA2 abolished the invasion induced by proNGF in cells DU145, PC3 and MDA-MB-231. These results indicate that EphA2 is involved in the invasion induced by proNGF of cells overexpressing EphA2.

### Analysis of the Expression of Genes in Breast Tumour Samples

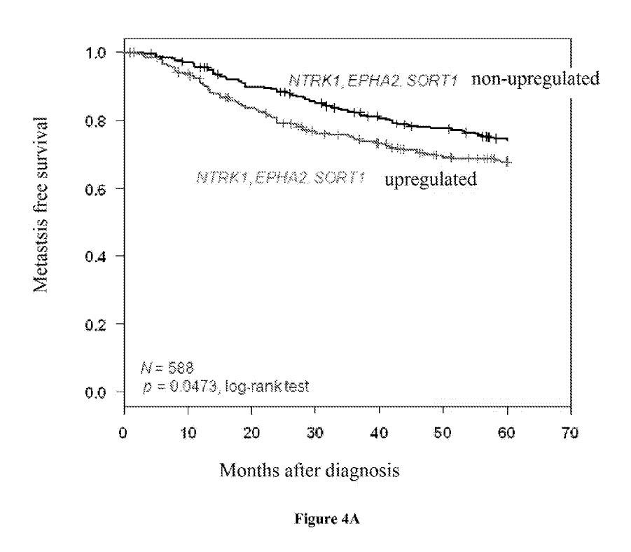

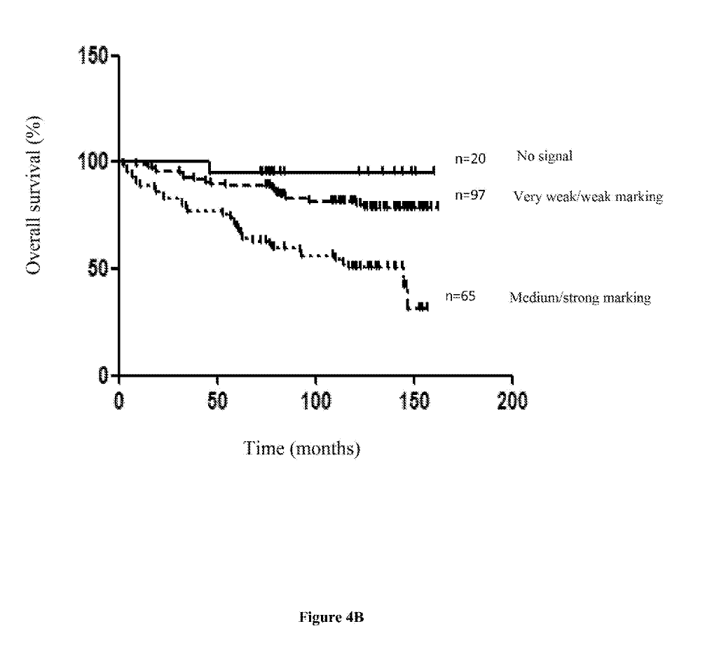

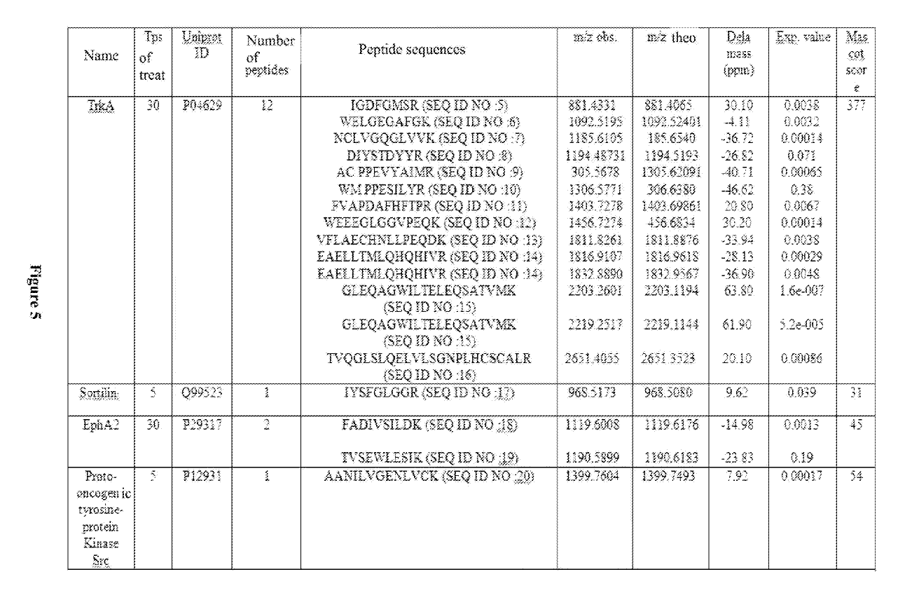

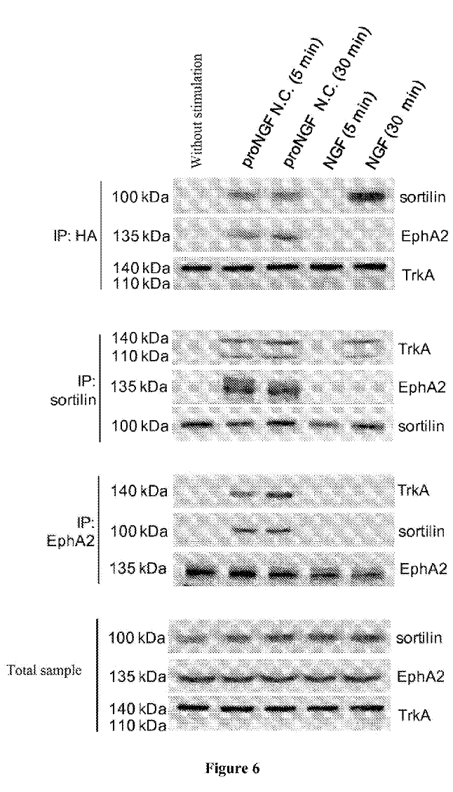

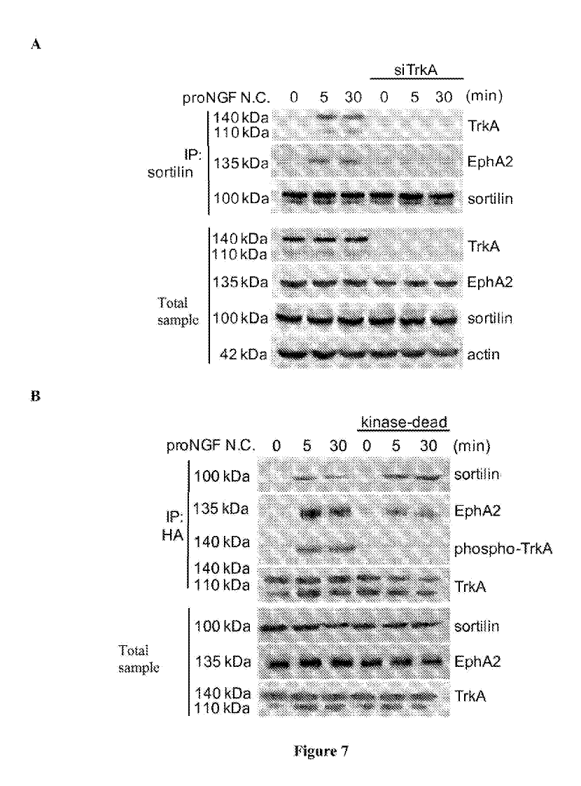

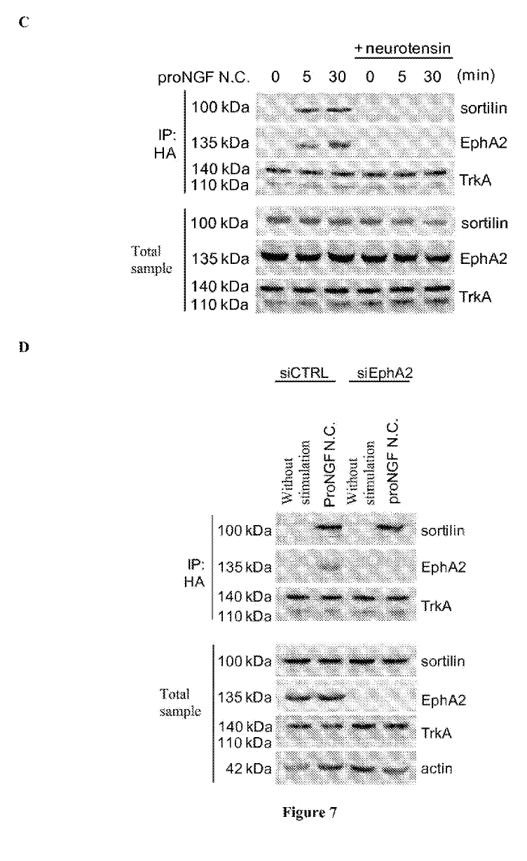

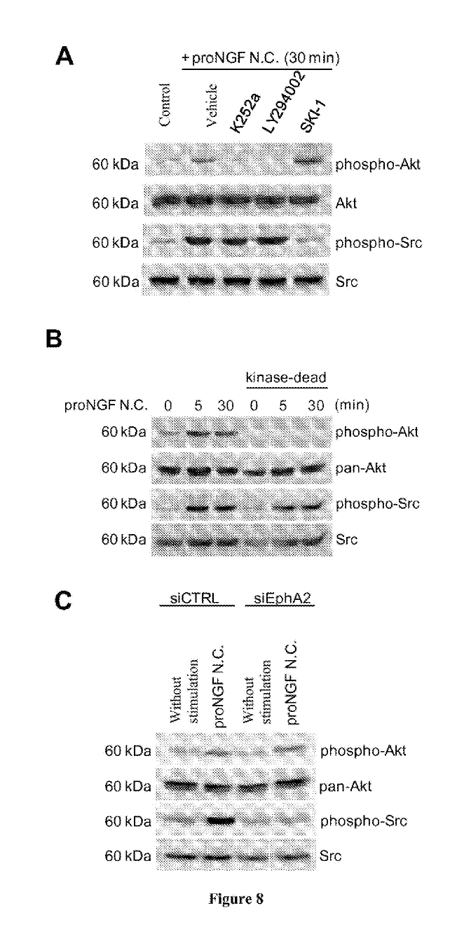

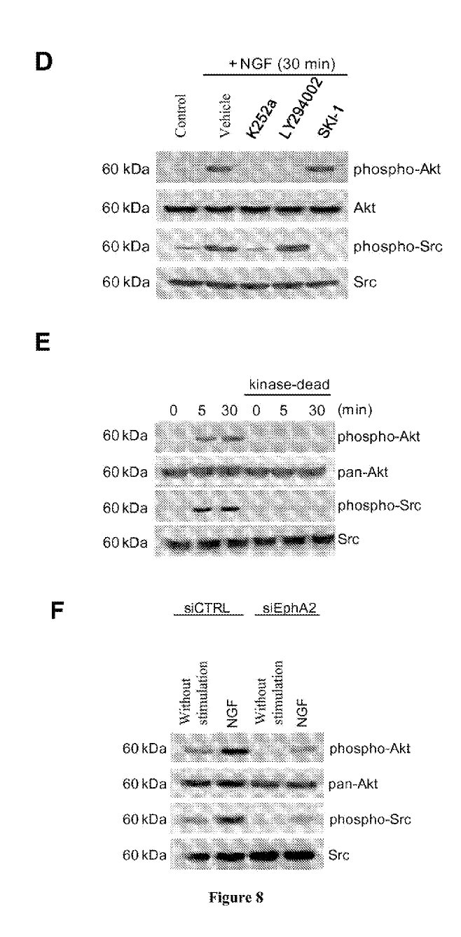

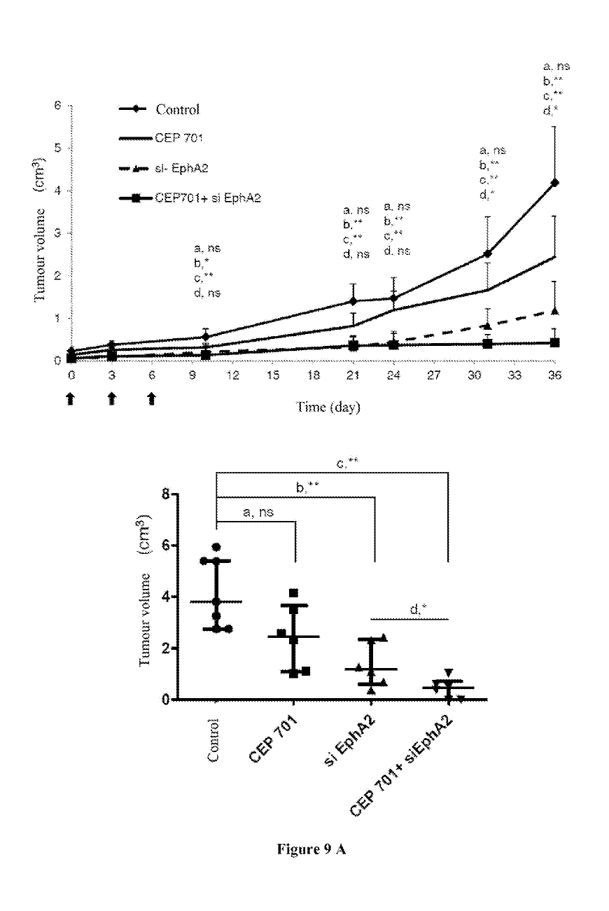

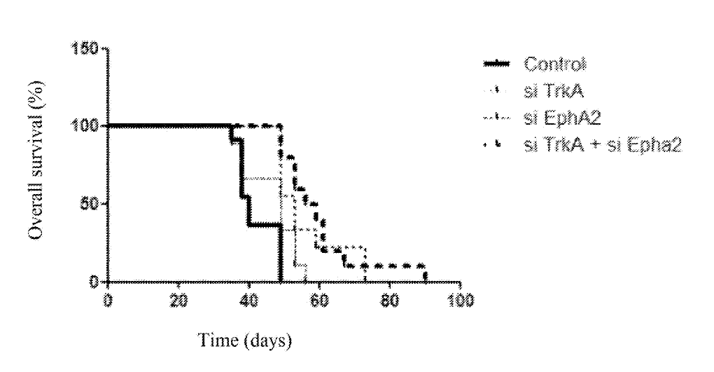

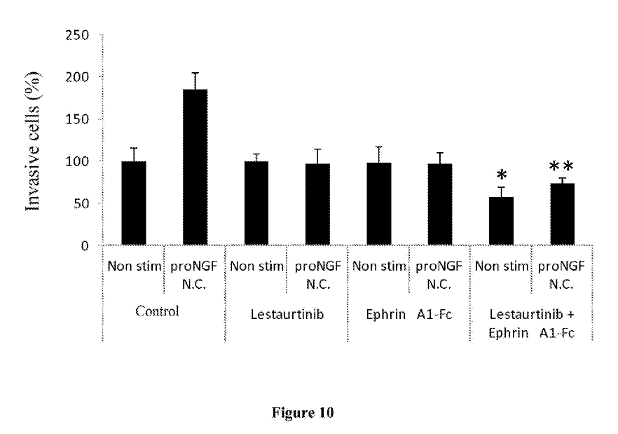

In addition, a retrospective analysis carried out on a cohort of 588 patients on DNA micro-arrays (DNA chips) made it possible to correlate the expression of the receptors sortilin, TrkA and EphA2 with a poor prognosis in breast cancer (FIG. 4 A). Indeed, the expression of the three receptors induced a significant reduction in the metastases free survival among the patients.

The detection of TrkA/EphA2 complexes in the breast tumours was also affected by using two microarray tissues (ref CBA4 (superbiochips) and ref Hbre-Duc 150Sur-01 (US Biomax)) i.e. a total of 182 tumour samples (n=182). The intensity of the PLA marking was scored as absence of signal, very weak/weak signal, medium/strong signal. The results are presented in the form of a Kaplan Meier graph. The results show that an association of the TrkA and EphA2 receptors revealed by the Duolink® technique is correlated with a significant reduction (log-rank test, p<0.0001) in the overall survival of patients. The detection of the TrkA/EphA2 complex is therefore correlated with a poor prognosis in breast cancer.

### Immunoprecipitation Tests

The formation of a complex between TrkA, EphA2 and sortilin was analysed by immunoprecipitation in cells MDA-MB-231 HA-TrkA as well as the PLA tests (Proximity Ligation Assay).

The association of the 3 receptors in the complex was first highlighted by mass spectrometry, the proteins immunoprecipitated by an antibody against HA-TrkA were identified (FIG. 5). The analysis by mass spectrometry made it possible to irrefutably demonstrate the presence of TrkA, sortilin and EphA2, in the complex formed by the action of proNGF.

The immunoprecipitation tests were carried out using antibodies against HA, against sortilin and against EphA2 in cells MDA-MB-231 HA-TrkA (FIG. 6).

In the absence of proNGF, no complex was observed between said three receptors. In contrast, with treatment by proNGF, sortilin and EphA2 were co-immunoprecipitated with TrkA.

By comparison, in the presence of NGF, the sortilin/TrkA Bond was only detected after 30 minutes of treatment, however the bond with EphA2 has not been observed, neither after 5 minutes of treatment nor after 30 minutes. These results have been confirmed by inverse immunoprecipitation of sortilin and EphA2. Hence, the NGF treatment appears to induce a delayed association of TrkA and sortilin but does not induce bonding with EphA2. These data confirm the results found in the literature, in that in the presence of NGF, sortilin acts as an endocytosis receptor in the neuron cell models.

### PLA Tests (Proximity Ligation Assay)

The PLA tests carried out on cells MDA-MB-231 were performed to confirm the interaction between sortilin and HA-TrkA and between HA-TrkA and EphA2 in cells MDA-MB-231 HA-TrkA (results not shown).

In the absence of proNGF, no PLA signal was displayed, which suggests that these receptors do not form a pre-existing complex.

In contrast, under treatment with proNGF, the PLA tests show an interaction between sortilin and TrkA and between EphA2 and TrkA, which indicates that TrkA interacts directly with sortilin and EphA2, and does so at a distance less than 40 nm (results not shown).

No PLA signal was detected in the tests between sortilin and EphA2, which suggests that proNGF does not introduce direct interaction between these two receptors (results not shown).

Moreover, the PLA tests in native cells MDA-MB-231, DU145, PC3 confirm that TrkA and EphA2 are engaged in a complex under treatment with proNGF, in the same way as cells MDA-MB-231 HA-TrkA (results not shown).

In comparison with proNGF, NGF induces no PLA signal in the cells MDA-MB-231, revealing the absence of receptor complex (results not shown).

### Sequential Invalidation Tests

The impact of a sequential invalidation of each of the receptors in the formation of this complex was then analysed.

The expression of TrkA was inhibited by interfering RNA (siTrkA) (FIG. 7A). TrkA and EphA2 are normally co-immunoprecipitated with sortilin after five minutes of proNGF treatment. In contrast, the cells treated with an anti-TrkA interfering RNA (siTrkA) did not show any association between EphA2 and TrkA with sortilin, which indicates that sortilin does not form a complex with EphA2 in the absence of TrkA.

Interestingly, in the cells which stably express kinase-dead TrkA and in which phosphorylation of TrkA has been abolished (FIG. 7B), proNGF continued to induce the association of TrkA with sortilin and EphA2. These results indicate that phosphorylation of TrkA is not necessary for the formation of TrkA/sortilin/EphA2 complex.

The involvement of sortilin in the sortilin/TrkA/EphA2 complex was then evaluated using neurotensin which inhibits by competition the bonding of proNGF to sortilin (FIG. 7C).

In the presence of neurotensin, TrkA precipitates neither sortilin nor EphA2. Thus, the proNGF/sortilin bonding would appear to be indispensable for the formation of the TrkA/sortilin/EphA2 complex.

Finally, the involvement of EphA2 in the association of the receptors complex has been studied using an anti-EphA2 interfering RNA (FIG. 7D).

The transient inhibition of EphA2 completely abolished the expression of this protein and consequently no bond was observed between EphA2 and TrkA.

Nevertheless, in the absence of EphA2, proNGF always induced an association between TrkA and sortilin. Consequently, TrkA associates with sortilin after treatment with proNGF in a manner independent of EphA2.

These results demonstrate the following points:

1. TrkA, sortilin and EphA2 do not form a pre-existing complex;

2. the TrkA/EphA2 requires the association of proNGF and sortilin as a prerequisite;

3. TrkA is necessary for the formation of this receptor complex, independently of the phosphorylation state thereof;

4. inhibition of EphA2 does not alter the TrkA/sortilin complex.

These results suggest that proNGF induces the association between sortilin and TrkA, then causes the recruitment of EphA2 on the sortilin/TrkA complex.

### Example 2

TrkA and EphA2 are involved differently in the activation of proteins Akt and Src, induced by proNGF.

Unless stated otherwise, the products, reagents and cell cultures are the same as those for example 1.

Proteins Akt and Src were both involved in the pro-invasive effect of proNGF. In order to identify the role of TrkA and EphA2 in the activation of proteins Akt and Src, cells MDA-MB-231 were treated respectively with a TrkA inhibitor (K252a), an anti-EphA2 siRNA (siEphA2) the sequence of which is presented in table 1, a PI3-K inhibitor (LY294002) (the activation of Akt in effect underlies that of PI3-K) and an inhibitor of Src (SKI-1).

The transfections of siRNA are produced by means of INTERFERin™ according to the manufacturers instructions (POL409-10, Polyplus transfection, Ozyme, Saint Quentin en Yvelines, France).

### RESULTS

As demonstrated in FIG. 8A, proNGF activated both Akt and Src.

Inhibition of the phosphorylation of TrkA through the use of K252a abolished the activation of Akt but not that of Src. Similar results were obtained through the use of a kinase-dead mutant of TrkA (FIG. 8B). This indicates that the activation of Akt, and not that of Src, is dependent on TrkA phosphorylation.

The inhibition of Akt activation using LY294002 does not modify the phosphorylation of Src. Similarly, the inhibition of Src with SKI-1 does not affect the phosphorylation of Akt. Consequently, the activation of Akt and Src by proNGF represents two different signalling channels.

The involvement of EphA2 in the activation of Akt and Src was determined by transient transfection of the cells having an EphA2 interference RNA (FIG. 8C). The inhibition of EphA2 reduced the phosphorylation of Src induced by proNGF, but not that of Akt.

It has therefore been shown that protein Akt is activated by proNGF via phosphorylation of TrkA, while protein Src is activated by EphA2 independently of the phosphorylation of TrkA

The effects of the NGF were determined for comparison (FIGS. 8D, E and F).

NGF increased the phosphorylations of Akt and Src, while the TrkA inhibitor, K252a, inhibited this activation (FIG. 8D). This result has been confirmed by the use of a Kinase-Dead mutant of TrkA (FIG. 8E). The activations of Src and Akt by NGF are not been connected as for proNGF (FIG. 8D). Unlike proNGF, the effects of NGF on Src and Akt are independent of EphA2 (FIG. 8F). The inhibition of EphA2 reduces only the base activation of phospho-Akt and phospho-Src but not the phosphorylation induced by the NGF. Indeed, the NGF is capable of increasing the phosphorylation of Akt and Src, even in the cells where the expression of EphA2 is inhibited. These results show that Akt and Src activation is downstream of TrkA in the presence of NGF.

These results also show that the activation of Src induced by proNGF requires both TrkA and EphA2, but that it is independent of the phosphorylation of TrkA. In contrast, the activation of Akt induced by proNGF is dependent on the phosphorylation of TrkA but not EphA2. Unlike proNGF, the signalling of NGF only requires TrkA in order to activate Akt and Src without any EphA2 involvement.

### Example 3

The TrkA/EphA2 complex is involved in the development and aggressiveness of in vivo tumours

Unless stated otherwise, the products, reagents and cell cultures were the same as those for example 1.

Tests of xenografts of tumour lines on immune deficient mice were carried out using cells MDA-MB-231.

### Xenografts of Tumour Lines in Immunodeficient Nice.

Experiment 1: the mice used were six-week old SCID females. Cells MDA-MB-231 HA-TrkA (3×10⁶) were injected subcutaneously into the flank of the mice. The mice with then randomly distributed into experimental groups (7 mice in the control group and 6 in the others). Fourteen days after inoculation with the cells, the animals were treated three times with an interval of 3 days between each injection. CEP-701 (Calbiochem) was dissolved in a mixture (40% polyethylene glycol 1000, 10% povidone C30 and 2% benzyl alcohol in distilled water) and injected by the intraperitoneal route (10 mg/kg). The EphA2 siRNA (7.5 microg/mouse) was injected close to the tumour mass using the in vivo jetPEI® (Polyplus transfection) according to the manufacturer's recommendations. The tumour volume was measured according to the following formula: π/6×length×width×(width+length)/2. The statistical analyses were carried out using the Mann and Whitney test and the GraphPad Prism 5.01 software.

Experiment 2: The mice used were six-week old SCID females. Cells MDA-MB-231 HA-TrkA (3×106) were injected subcutaneously into the flank of the mice. The mice were then randomly distributed into the experimental groups (10 mice/group). Fourteen days after inoculation with the cells, the animals were treated five times with an interval of 3 days between each injection. The EphA2 and TrkA siRNA (7.5 microg/mouse) were injected close to the tumour mass using the in vivo jetPEI® (Polyplus transfection)according to the manufacturer's recommendations. The tumour volume was measured according to the following formula: π/6×length×width×(width+length)/2. Survival is presented in the form of a Kaplan Meier graph.

### RESULTS

It had previously been demonstrated that K252a (TrkA inhibitor) reduces the growth of tumours xenografted with cells MDA-MB-231 overexpressing TrkA (34).

Here, a lesser dose of analogue of K252a (CEP-701 at 10 mg/kg) was used in order to guarantee a moderate reduction in tumour growth.

As shown in FIGS. 9 A and B, the tumour volume is slightly, but not significantly, reduced by the treatment with CEP-701 when compared with the control. The targeting of EphA2 also strongly reduces the growth of xenografted tumours.

In a particularly interesting way, the combined CEP-701/siEphA2 treatment induced a significant reduction in the tumour load in comparison to CEP-701 only or siEphA2 only. These results indicate that TrkA and EphA2 cooperate in vivo in order to increase tumour growth.

In order to validate the involvement of TrkA in tumour development, a second animal experiment was carried out by replacing CEP-701 with an siRNA against TrkA, which specifically invalidated its expression. The results are shown in terms of survival. The median survivals are respectively 40 days in the control, 53 days in the presence of siTrkA, 49 days in the presence of siEphA2 and 57.5 days in the presence of siTrkA and siEphA2. These results are significantly different as indicated by the log-rank test (P=0.0022). These results indicate that CEP-701 and an siRNA against TrkA have the same effects on tumour growth and confirm the observations made by the western blot method on tumour cells. In total, the data also shows that the inhibition of growth observed by the treatment with CEP-701 depends on its effects on the TrkA receptor.

### Example 4

A treatment combining a TrkA inhibitor (lestaurtinib) and an EphA2 inhibitor (EphrinA1-Fc) reduces the invasion of breast cancer cells

Unless stated otherwise, the products, reagents and cell cultures were the same as those for example 1.

The invasion test was carried out on the cells MDA-MB-231. Non-stimulated cells of proNGF served as the control and determine a base invasion of 100% (white bar in FIG. 10). The following concentrations were applied for the treatments: 0.5 nM proNGF N.C., 10 nM lestaurtinib, Ephrin A1-Fc (1 microg/ml), DMSO 1/1000 th vol/vol for non-stimulated conditions (Non stim). For statistics, the error bars represent the standard deviation. * p<0.001 for the combination of lestaurtinib+Ephrin A1-Fc treatment vs. the individual non-stimulated treatments (lestaurtinib or Ephrin A1-Fc). ** p<0.0065 for the lestaurtinib+Ephrin A1-Fc combination treatment vs. the treatments with lestaurtinib in the presence of proNGF; p<0.002 for the lestaurtinib+Ephrin A1-Fc combination treatment vs. the treatments with Ephrin A1-Fc in the presence of proNGF.

### RESULTS

As can be seen in FIG. 10, the lestaurtinib-Ephrin A1-Fc combination significantly reduces cell invasion and is more effective than the treatments in which a single inhibitor is used (lestaurtinib only or Ephrin A1-Fc only).

### Example 5

The TrkA/EphA2 complex is also found in several types of tumour.

Unless stated otherwise, the products, reagents and cell cultures were the same as those for example 1.

Proximity Ligation Assay on Paraffined Tumour Samples from the Buccal Cavity.

The paraffin is removed from the stage T4 buccal cavity tumour samples (N0, M0) with bone infiltration (obtained from the anatomopathology department of the CHRU in Lille) in baths of ClaRal (derivative of xylene) (for 1×12 h and 1×5 h). The sections are then rehydrated by successive baths: ClaRal/ethanol 100% (1:1) (1×5 min), ethanol 100% (2×5 min), ethanol 96% (2×5 min), ethanol 70% (2×5 min), distilled water (1×5 min). The blocking of endogenous peroxidases is achieved by a solution of hydrogen peroxide supplied in the Duolink kit, In Situ® Detection Reagents Brightfield (DU092012; Sigma-Aldrich) 10 min at 20° C. The sections are then washed with TBS (20 mM Tris base, 137 mM NaCl) (2×5 min) then incubated with a blocking solution supplied in the Duolink® kit (Blocking solution; 1 h; 20° C.). The samples are then incubated, the primary antibodies anti-TrkA diluted to 1/25 th (ANT-018; Alomone Labs) and anti-EphA2 diluted to 1/50 th (AF3035; RD systems), in the blocking solution at 4° C. overnight. The sections are then washed three times with solution A (0.01 M Tris, 0.15 M NaCl, 0.05% Tween 20) for 5 minutes and then incubated for 1 hour at 37° C. with the PLA probes: oligo-marked anti-rabbit “plus” (DU092006; Sigma-Aldrich) and anti-goat “minus” (DU092002; Sigma-Aldrich) diluted to ⅕ th in a solution of PBS 1× 1% SVP (Phosphate Buffered Saline: 2.7 mM KCl, 8 mM Na2HPO4, 1.8 mM KH2PO4, 137 mM NaCl, pH 7.4; 1% foetal calf serum). The ligation step is carried out according to the manufacturer's recommendations. After two washes of 5 minutes with solution A, the probes were amplified using a polymerase ( 1/80 th in amplification buffer 1×; 2 hours at 37° C.). The signal detection and the counter staining are carried out according to the protocol established by the supplier. The sections are then washed three times for 5 minutes in distilled water and then dehydrated by successive ethanol baths 70% (2×5 min), ethanol 96% (2×5 min), ethanol 100% (2×5 min), ClaRal/ethanol 100% (1:1) (1×5 min) and ClaRal (2×10 min). The slides are dried and then mounting between the slide and cover is carried out using Duolink® mounting medium (DU092012; Sigma-Aldrich). The PLA images (brown points) are obtained using a microscope coupled to a camera (lens 40×, microscope Eclipse Ti; Nikon, France) then analysed using the NIS-Elements BR software from Nikon. A brown point reflects an interaction between two proteins (separation of less than 40 nm; quantitative method).

### RESULTS

The expression of TrkA and EphA2 was detected by immunohistochemical staining and TrkA/EphA2 association was detected by PLA (Duolink®) in a buccal cavity tumour. Although the adjacent epithelium and the tumour masses showed a TrkA and EphA2 marking, only the tumour mass was positive (dot-shaped marking) for the PLA (results not shown). This demonstrates an action mechanism of TrkA and EphA2 in the buccal cavity tumours similar to that in the breast tumours.

It has therefore been shown by in vitro and in vivo experiments that TrkA and EphA2 have an effect on the invasion and growth of cancer cells, justifying the interest in combining a TrkA inhibitor and an EphA2 inhibitor in order to combat these forms of solid cancer.

### REFERENCES

(1) Magné N, Chargari C, Conforti R, Toillon R A, Bauduceau O, Védrine L, Khayat D, Spano J P. [Mechanisms of resistance to molecular targeted therapies in breast cancer: update and future]. Bull Cancer. 2010 March; 97(3):385-95.
(2) Adriaenssens E, Vanhecke E, Saule P, Mougel A, Page A, Romon R, Nurcombe V, Le Bourhis X, Hondermarck H. Nerve growth factor is a potential therapeutic target in breast cancer. Cancer Res. 2008 Jan. 15; 68(2):346-51.
(3) Demont Y, Corbet C, Page A, Ataman-Ônal Y, Choquet-Kastylevsky G, Fliniaux I, Le Bourhis X, Toillon R A, Bradshaw R A, Hondermarck H. Pro-nerve growth factor induces autocrine stimulation of breast cancer cell invasion through tropomyosin-related kinase A (TrkA) and sortilin protein. J Biol Chem. 2012 Jan. 13; 287(3): 1923-31.
(4) U.S. National Institutes of Health, study NCT00079482), Levis M, Ravandi F, Wang E S, Baer M R, Perl A, Coutre S, Erba H, Stuart R K, Baccarani M, Cripe L D, Tallman M S, Meloni G, Godley L A, Langston A A, Amadori S, Lewis I D, Nagler A, Stone R, Yee K, Advani A, Douer D, Wiktor-Jedrzejczak W, Juliusson G, Litzow M R, Petersdorf S, Sanz M, Kantarjian H M, Sato T, Tremmel L, Bensen-Kennedy D M, Small D, Smith B D. Results from a randomized trial of salvage chemotherapy followed by lestaurtinib for patients with FLT3 mutant AML in first relapse. Blood. 2011 Mar 24; 117(12):3294-301
(5) Trk kinase inhibitors as new treatments for cancer and pain, Tao Wang, Dingwei Yu & Michelle L Lamb, 2009 Informa UK Ltd ISSN 1354-3776 Cancer Discovery, AstraZeneca R&D Boston, Waltham, Mass., USA;
(6) Angiostatic effects of K252a, a Trk inhibitor, in murine brain capillary endothelial cells, Arien-Zakay H, Kohan M, Lelkes P I, 2010 June;339 (1-2):201-13. doi: 10.1007/s11010-010-0386-9.
(7) Ruggeri B, Miknyoczki S, Singh J, et al. Role of neurotrophin-Trk interactions in oncology: the anti-tumor efficacy of potent and selective Trk tyrosine kinase inhibitors in pre-clinical tumor models. Curr Med Chem 1999; 6:845-57
(8) Cee V J, Albrecht B K, Geuns-Meyer S, et al. Alkynylpyrimidine amide derivatives as potent, selective, and orally active inhibitors of Tie-2 kinase. J Med Chem 2007; 50:627-40;
(9) Hudkins R L, Josef K A. Pyrazole Trk kinase inhibitors for the treatment of cancer. Expert Opin Ther Patents 2007; 17:351-6;
(10) Das J, Furch J A, Liu C, et al. Discovery and SAR of 2-amino-5-(thioaryl)thiazoles as potent and selective Itk inhibitors. Bioorg Med Chem Lett 2006; 16:3706-12;
(11) Kim S H, Tokarski J S, Leavitt K J, et al. Identification of 2-amino-5-(thioaryl)thiazoles as inhibitors of nerve growth factor receptor TrkA. Bioorg Med Chem Lett 2008;18:634-9
(12) Ghose A K, Herbertz T, Pippin D A, et al. Knowledge based prediction of ligand binding modes and rational inhibitor design for kinase drug discovery. J Med Chem 2008; 51:5149-71
(13) Gingrich D E, Yang S X, Gessner G W, et al. Synthesis, modeling, and in vitro activity of (3′ S)-epi-K-252a analogues. Elucidating the stereochemical requirements of the 3′-sugar alcohol on TrkA tyrosine kinase activity. J Med Chem 2005; 48:3776-83.
(14) Tripathy R, Angeles T S, Yang S X, et al. TrkA kinase inhibitors from a library of modified and isosteric staurosporine aglycone. Bioorg Med Chem Lett 2008; 18:3551-5
(15) Wood E R, Kuyper L, Petrov K G, et al. Discovery and in vitro evaluation of potent TrkA kinase inhibitors: oxindole and aza-oxindoles. Bioorg Med Chem Lett 2004; 14:953-71
(16) Faivre S, Demetri G, Sargent W, et al. Molecular basis for sunitinib efficacy and future clinical development. Nat Rev Drug Discov 2007; 6:734-45
(17) Fancelli D, Moll J, Varasi M, et al. 1,4,5,6-tetrahydropyrrolo[3,4-c]pyrazoles: Identification of a potent aurora kinase inhibitor with a favorable antitumor kinase inhibition profile. J Med Chem 2006; 49:7247-51
(18) Lippa B, Morris J, Corbett M, et al. Discovery of novel isothiazole inhibitors of the TrkA kinase: structure-activity relationship, computer modeling, optimization, and identification of highly potent antagonists. Bioorg Med Chem Lett 2006; 16:3444-8
(19) Albaugh et al. (2012) ACS Med. Chem. Lett., 3(2): 140-145
(20) Kohler et al. Nature. 1975; 256(5517):495-7
(21) Cote et al Proc Natl Acad Sei USA. 1983; 80(7):2026-30
(22) Tuerk et al. (1990) Science, 249, 505-510
(23) Ljubica Ivanisevic, THE JOURNAL OF BIOLOGICAL CHEMISTRY VOL. 282, 5 NO. 23, pp. 16754-16763, Jun. 8, 2007
(24) Phase II Study of Dasatinib (BMS-354825) in Patients With Metastatic Adenocarcinoma of the Pancreas, Cheng Ean Chee, Smitha Krishnamurthi, Charles J. Nock et al. The Oncologist 2013; 18:1091-1092
(25) Enhancing Chemotherapy Response with Sustained EphA2, Silencing Using Multistage Vector Delivery, Haïfa Shen, Cristian Rodriguez-Aguayo, Rong

Xu et al., Clin Cancer Res 2013; 19:1806-1815
(26) Katherine R. Amato J Clin Invest. 2014; 124(5):2037-2049
(27) Zelinski D P, Zantek N D, Stewart J C, Irizarry A R, Kinch M S. EphA2 overexpression causes tumorigenesis of mammary epithelial cells. Cancer Res. 2001; 61:2301-6.

(28) Hochgrafe F, Zhang L, O'Toole S A, Browne B C, Pinese M, Porta Cubas A, et al. Tyrosine phosphorylation profiling reveals the signaling network characteristics of Basal breast cancer cells. Cancer Res. 2010; 70:9391-401.

(29) Drake J M, Graham N A, Stoyanova T, Sedghi A, Goldstein A S, Cai H, Smith D A, Zhang H, Komisopoulou E, Huang J, Graeber T G, Witte O N. Oncogene-specific activation of tyrosine kinase networks during prostate cancer progression. Proc Natl Acad Sei USA. 2012 Jan. 31; 109(5):1643-8
(30) Batson J, Maccarthy-Morrogh L, Archer A, Tanton H, Nobes C D. EphA receptors regulate prostate cancer cell dissemination through Vav2-RhoA mediated cell-cell repulsion. Biol Open. 2014 May 2.
(31) Kotoula V, Lambaki S, Televantou D, Kalogera-Fountzila A, Nikolaou A, Markou K, Misailidou D, Syrigos K N, Fountzilas G. STAT-Related Profiles Are Associated with Patient Response to Targeted Treatments in Locally Advanced SCCHN. Transl Oncol. 2011 Feb. 1; 4(1):47-58
(32) Bioorganic & Medicinal Chemistry Letters, Volume 8, Issue 16, 18 Aug. 1998, pages 2219-2222
(33) M. Egholm et al., J. Am. Chem. Soc. (1992), 114, 1895-1897
(34) Maleck, L., et al., 1994, Methods in Molecular Biology, 28, ch 36, Ed P. G. Isaac, Humana Press, Inc., Totowa, N I
(35) Lagadec C, Meignan S, Adriaenssens E, Foveau B, Vanhecke E, Romon R, et al. TrkA overexpression enhances growth and metastasis of breast cancer cells. Oncogene. 2009; 28:1960-70.
(36) Wykosky J, Debinski W. The EphA2 receptor and ephrinA1 ligand in solid tumors: function and therapeutic targeting. Mol Cancer Res. 2008 Dec; 6(12): 1795-806.

(37) Arrighi N, Bodei S, Zani D, Simeone C, Cunico SC, Missale C, Spano P, Sigala S. Nerve growth factor signaling in prostate health and disease. Growth Factors. 2010 June; 28(3):191-201
(38) Anagnostopoulou V, Pediaditakis I, Alkahtani S, Alarifi S A, Schmidt E M, Lang F, Gravanis A, Charalampopoulos I, Stoumaras C. Differential effects of dehydroepiandrosterone and testosterone in prostate and colon cancer cell apoptosis: the role of nerve growth factor (NGF) receptors. Endocrinology. 2013 July; 154(7):2446-56
(39) Kolokythas A, Cox D P, Dekker N, Schmidt B L. Nerve growth factor and tyrosine kinase A receptor in oral squamous cell carcinoma: is there an association with perineural invasion? J Oral Maxillofac Surg. 2010 June; 68(6):1290-5.
(40) Trovisco V, Soares P, Preto A, Castro P, Maximo V, Sobrinho-Simões M. Molecular genetics of papillary thyroid carcinoma: great expectations. Arq Bras Endocrinol Metabol. 2007 July; 51(5):643-53.
(41) Karidis N P, Giaginis C, Tsourouflis G, Alexandrou P, Delladetsima I, Theocharis S. Eph-A2 and Eph-A4 expression in human benign and malignant thyroid lesions: an immunohistochemical study. Med Sci Monit. 2011 September; 17(9):BR257-65
(42) Zhu Z, Friess H, diMola F F, Zimmermann A, Graber H U, Korc M, Büchler M W. Nerve growth factor expression correlates with perineural invasion and pain in human pancreatic cancer. J Clin Oncol. 1999 August; 17(8):2419-28.
(43) Nakagawara A, Arima-Nakagawara M, Scavarda N J, Azar C G, Cantor A B, Brodeur G M. Association between high levels of expression of the TRK gene and favorable outcome in human neuroblastoma. N Engl J Med. 1993 Mar. 25; 328(12):847-54.
(44) Wang Y, Hagel C, Hamel W, Müller S, Kluwe L, Westphal M. Trk A, B, and C are commonly expressed in human astrocytes and astrocytic gliomas but not by human oligodendrocytes and oligodendroglioma. Acta Neuropathol. 1998 October; 96(4):357-64.
(45) Truzzi F, Marconi A, Lotti R, Dallaglio K, French L E, Hempstead B L, Pincelli C. Neurotrophins and their receptors stimulate melanoma cell proliferation and migration. J Invest Dermatol. 2008 August; 128(8):2031-40
(46) Kim M S, Kim G M, Choi Y J, Kim H J, Kim Y J, Jin W. c-Src activation through a TrkA and c-Src interaction is essential for cell proliferation and hematological malignancies. Biochem Biophys Res Commun. 2013 Nov. 15; 441(2):431-7.
(47) Lazarova P, Wu Q, Kvalheim G, Suo Z, Haakenstad K W, Metodiev K, Nesland J M. Growth factor receptors in hematopoietic stem cells: EPH family expression in CD34+ and CD133+ cell populations from mobilized peripheral blood. Int J Immunopathol Pharmacol. 2006 January-March; 19(1):49-56.
(48) Irizarry R A, Hobbs B, Collin F, Beazer-Barclay Y D, Antonellis K J, Scherf U, et al. Exploration, normalization, and summaries of high density oligonucleotide array probe level data. Biostatistics. 2003; 4:249-64.
(49) Parker J S, Mullins M, Cheang M C, Leung S, Voduc D, Vickery T, et al. Supervised risk predictor of breast cancer based on intrinsic subtypes. J Clin Oncol. 2009; 27:1160-7.
(50) McShane L M, Altman D G, Sauerbrei W, Taube S E, Gion M, Clark G M. REporting recommendations for tumor MARKer prognostic studies (REMARK). Breast Cancer Res Treat. 2006; 100:229-35.
(51) Zhu Z, Kleeff J, Kayed H, Wang L, Korc M, Buchler M W, et al. Nerve growth factor and enhancement of proliferation, invasion, and tumorigenicity of pancreatic cancer cells. Mol Carcinog. 2002; 35:138-47.
(52) Festuccia C, Muzi P, Gravina G L, Millimaggi D, Speca S, Dolo V, et al. Tyrosine kinase inhibitor CEP-701 blocks the NTRK1/NGF receptor and limits the invasive capability of prostate cancer cells in vitro. Int J Oncol. 2007; 30:193-200.
(53) Truzzi F, Marconi A, Lotti R, Dallaglio K, French L E, Hempstead B L, et al. Neurotrophins and their receptors stimulate melanoma cell proliferation and migration. J Invest Dermatol. 2008; 128:2031-40.
(54) Kolokythas A, Cox D P, Dekker N, Schmidt B L. Nerve growth factor and tyrosine kinase A receptor in oral squamous cell carcinoma: is there an association with perineural invasion? J Oral Maxillofac Surg. 2010; 68:1290-5.
(55) R&D systems, Recombinant Human Ephrin-A1 Fe Chimera) (Mark S. Duxbury, Biochemical and Biophysical Research Communications 320 (2004) 1096-1102
(56) Munetaka Hirose, British Journal of Pharmacology (2004) 142, 222-228

1. A method of treating a solid cancer in a patient treated with a TrkA inhibitor, comprising the step of administering to said patient a EphA2 inhibitor, wherein said TrkA inhibitor is a TrkA antagonist, an inhibitor of TrkA receptor expression, an inhibitor of TrkA tyrosine kinase activity or a molecule preventing the binding of signaling adapter molecules, and wherein said EphA2 inhibitor is an EphA2 antagonist, an inhibitor of EphA2 receptor expression or a molecule preventing the binding of signaling adapter molecules.

2. A method of treating a solid cancer in a patient treated with an EphA2 inhibitor, comprising the step of administering to said patient a TrkA inhibitor, wherein said TrkA inhibitor is a TrkA antagonist, an inhibitor of TrkA receptor expression, an inhibitor of TrkA tyrosine kinase activity or a molecule preventing the binding of signaling adapter molecules, and wherein said EphA2 inhibitor is an EphA2 antagonist, an inhibitor of EphA2 receptor expression or a molecule preventing the binding of signaling adapter molecules.

3. A pharmaceutical composition comprising a TrkA inhibitor, an EphA2 inhibitor and at least one pharmaceutically acceptable carrier, wherein said TrkA inhibitor is a TrkA antagonist, an inhibitor of TrkA receptor expression, an inhibitor of TrkA tyrosine kinase activity or a molecule preventing the binding of signaling adapter molecules, and wherein said EphA2 inhibitor is an EphA2 antagonist, an inhibitor of EphA2 receptor expression, or a molecule preventing the binding of signaling adapter molecules.

4. (canceled)

5. A combination product comprising:
a TrkA inhibitor, and
an EphA2 inhibitor,
wherein said TrkA inhibitor is a TrkA antagonist, an inhibitor of TrkA receptor expression, an inhibitor of TrkA tyrosine kinase activity or a molecule preventing the binding of signaling adapter molecules, and wherein said EphA2 inhibitor is an EphA2 antagonist, an inhibitor of EphA2 receptor expression or a molecule preventing the binding of signaling adapter molecules.

6. (canceled)

7. A method for prognosis of the survival of a patient suffering from a solid cancer, detecting the expression of TrkA and EphA2 in a biological sample of the patient, the co-expression of TrkA and EphA2 being associated with a poor prognosis of survival of the patient.

8. The method according to claim 7, in which the step of detecting the expression of TrkA and EphA2 is carried out by detecting a TrkA/EphA2 complex.

9. The method according to claim 7, in which the biological sample comes from a biopsy of a tumor of the patient,

10-12. (canceled)

13. The method according to claim 1, wherein said solid cancer is breast cancer, prostate cancer, colon cancer, tongue cancer, cancer of the oropharyngeal sphere, thyroid cancer, pancreatic cancer, neuroblastoma, glioma or skin cancer

14. The method according to claim 1, wherein said solid cancer is breast cancer. prostate cancer or cancer of the oropharyngeal sphere.

15. The method according to claim 1, wherein said cancer is breast cancer.

16. The method according to claim 2, wherein said solid cancer is breast cancer, prostate cancer, colon cancer, tongue cancer, cancer of the oropharyngeal sphere, thyroid cancer, pancreatic cancer, neuroblastoma, glioma or skin cancer

17. The method according to claim 2, wherein said solid cancer is breast cancer, prostate cancer or cancer of the oropharyngeal sphere.

18. The method according to claim 2, wherein said cancer is breast cancer.

19. The method according to claim 7, wherein said solid cancer is breast cancer, prostate cancer, colon cancer, tongue cancer, cancer of the oropharyngeal sphere, thyroid cancer, pancreatic cancer, neuroblastoma, glioma or skin cancer.

20. The method according to claim 7, wherein said solid cancer is breast cancer, prostate cancer or cancer of the oropharyngeal sphere.

21. The method according to claim 7, wherein said solid cancer is breast cancer.

22. The method of treating a solid cancer, comprising administering a TrkA inhibitor and an EphA2 inhibitor to a patient in need thereof.

23. The method according to claim 22, wherein said TrkA inhibitor is a TrkA antagonist, an inhibitor of TrkA receptor expression, an inhibitor of TrkA tyrosine kinase activity or a molecule preventing the binding of signaling adapter molecules.

24. The method according to claim 22, wherein said EphA2 inhibitor is an EphA2 antagonist, an inhibitor of EphA2 receptor expression or a molecule preventing the binding signaling adapter molecules.

25. The method according to claim 22, wherein said solid cancer is breast cancer, prostate cancer, colon cancer tongue cancer, cancer of the oropharyngeal sphere, thyroid cancer, pancreatic cancer, neuroblastoma, glioma or skin cancer

26. The method according to claim 22, wherein said solid cancer is breast cancer, prostate cancer or cancer of the oropharyngeal sphere.

27. The method according to claim 22, wherein said solid cancer is breast cancer.
